(19) United States
(12) Patent Application Publication
(10) Pub. No.: US 2025/0090794 A1
(43) Pub. Date: Mar. 20, 2025

(54) SLEEP MANAGEMENT SYSTEM AND SLEEP MANAGEMENT METHOD

(71) Applicant: SAMSUNG ELECTRONICS CO., LTD., Suwon-si (KR)

(72) Inventors: Chanwon LEE, Suwon-si (KR); Doyoon KIM, Suwon-si (KR)

(73) Assignee: SAMSUNG ELECTRONICS CO., LTD., Suwon-si (KR)

(21) Appl. No.: 18/783,016

(22) Filed: Jul. 24, 2024

Related U.S. Application Data
(63) Continuation of application No. PCT/KR2024/008646, filed on Jun. 21, 2024.

(30) Foreign Application Priority Data
Sep. 15, 2023 (KR) 10-2023-0123500

(51) Int. Cl. A61M 21/02; A61M 21/00; G16H 40/63

(52) U.S. Cl. CPC A61M 21/02; G16H 40/63; A61M 2021/0083; A61M 2205/3303; A61M 2205/3584

(57) ABSTRACT

A sleep management system includes: receiving, by a hub device, first data collected by a first sensor and second data collected by a second sensor; obtaining, by the hub device, first processed data by processing the first data and second processed data by processing the second data; transmitting, by the hub device, the first processed data and the second processed data to a user device; obtaining, by the user device, sleep state information by inputting the first processed data and the second processed data to a machine learning model of the user device, wherein the sleep state information is associated with a sleep state of a user; and transmitting, by the user device, the sleep state information to a server device.

### CROSS-REFERENCE TO RELATED APPLICATIONS

This application is a by-pass continuation application of International Application No. PCT/KR2024/008646, filed on Jun. 21, 2024, which is based on and claims priority to Korean Patent Application No. 10-2023-0123500, filed on Sep. 15, 2023, in the Korean Intellectual Property Office, the disclosures of which are incorporated by reference herein their entireties.

### BACKGROUND

### 1. Field

The disclosure relates to a sleep management system and a sleep management method.

### 2. Description of Related Art

Sleep is an essential factor for a person's health and well-being as it plays a variety of roles, including repairing the body, forming new memories, staying focused, and removing waste products accumulated in the brain. Maintaining quality sleep is very important for humans to live healthy and smoothly.

To maintain quality sleep, there is a growing demand for polysomnography, a test for detecting diseases or sleep disorders that occur during sleep, but the polysomnography is expensive, takes a long time and is performed with various sensors attached to the body, thereby causing discomfort to a subject (a person).

For this reason, various devices capable of analyzing the person's sleep state have been being developed, but the devices have remarkably low accuracy in measuring the sleep state as compared to the polysomnography. Thus, there is a need for devices and methods with higher accuracy in measuring the person's sleep state.

### SUMMARY

Provided are a device and a method for measuring a person's sleep state in everyday life even without various sensors attached to the body. Provided are a device and a method for analyzing the person's sleep state in a user device to avoid a violation of privacy. Provided are a device and a method for processing data by multiple devices, thus an individual device's data processing burden can be reduced.

According to an aspect of the disclosure, values output by a machine learning model of a hub device may be used as input values to a machine learning model of a user device, to obtain accurate data related to the user's sleep state. According to an aspect of the disclosure, quality sleep of the user may be induced.

According to an aspect of the disclosure, a sleep management system includes: a plurality of sensors configured to collect data of a user; a hub device configured to process the data collected from the plurality of sensors; a user device configured to obtain sleep state information by processing the data processed by the hub device, wherein the sleep state information is associated with a sleep state of the user; and a server device configured to control at least one home appliance, based on the sleep state information obtained by the user device, wherein the plurality of sensors include a first sensor configured to collect first data and a second sensor configured to collect second data, wherein the hub device is further configured to: receive the first data and the second data, obtain first processed data by processing the first data, obtain second processed data by processing the second data, and transmit the first processed data and the second processed data to the user device, and wherein the user device is further configured to: receive the first processed data and the second processed data from the hub device, obtain the sleep state information by inputting the first processed data and the second processed data to a machine learning model of the user device, and transmit the sleep state information to the server device.

According to an aspect of the disclosure, a sleep management method includes: receiving, by a hub device, first data collected by a first sensor and second data collected by a second sensor; obtaining, by the hub device, first processed data by processing the first data and second processed data by processing the second data; transmitting, by the hub device, the first processed data and the second processed data to a user device; obtaining, by the user device, sleep state information by inputting the first processed data and the second processed data to a machine learning model of the user device, wherein the sleep state information is associated with a sleep state of a user; and transmitting, by the user device, the sleep state information to a server device.

According to an aspect of the disclosure, a non-transitory storage medium is configured to store computer-readable instructions, wherein the instructions, which enable, when executed by a processor, the processor to receive, through a communicator, processed data processed by a hub device from the hub device which processes data of a user collected from a plurality of sensors; obtain sleep state information associated with a sleep state of the user by inputting the processed data to a machine learning model; and control at least one home appliance based on the sleep state information.

According to an aspect of the disclosure, a person's sleep state may be accurately measured in everyday life even without various sensors attached to the body. According to an aspect of the disclosure, the person's sleep state may be analyzed in a user device to avoid a violation of privacy.

According to an aspect of the disclosure, data is processed by multiple devices, so that the level of data processing burden that each device bears may be reduced. According to an aspect of the disclosure, values output by a machine learning model stored in a hub device may be used as input values to a machine learning model stored in a user device, to obtain accurate data related to the user's sleep state. According to an aspect of the disclosure, quality sleep of the user may be induced.

The effects according to the disclosure are not limited thereto, and throughout the specification, those of ordinary skill in the art would understand that there may be other effects unmentioned

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 illustrates an example of a network of a sleep management system, according to an embodiment;

FIG. 2 illustrates an example of a structure of a sleep management system, according to an embodiment;

FIG. 3 illustrates an example of a control block diagram of a sleep management system, according to an embodiment;

FIGS. 4 and 5 illustrate an example of a plurality of sensors of a sleep management system, according to an embodiment;

FIG. 6 illustrates an example of a flowchart of a sleep management system, according to an embodiment;

FIG. 7 illustrates a procedure for processing data collected from a plurality of sensors of a sleep management system, according to an embodiment;

FIG. 8 illustrates sleep stages;

FIG. 9 illustrates an example of sleep state information derived by a sleep management system, according to an embodiment;

FIG. 10 illustrates an example of sleep summary information derived by a sleep management system, according to an embodiment;

FIGS. 11 and 12 illustrate an example of a flowchart of a sleep management method, according to an embodiment;

FIG. 13 illustrates an example of a preset operation to induce sleep of the user;

FIG. 14 illustrates an example of a preset operation to relieve stress of the user;

FIG. 15 illustrates an example of a preset operation to relieve sleep disorders of the user; and

FIG. 16 illustrates an example of a preset operation to induce wakeup of the user.

### DETAILED DESCRIPTION

It is understood that one or more embodiments of the disclosure and associated terms are not intended to limit technical features herein to particular embodiments, but encompass various changes, equivalents, or substitutions. Like reference numerals may be used for like or related elements throughout the drawings. The singular form of a noun corresponding to an item may include one or more items unless the context states otherwise.

Throughout the specification, “A or B”, “at least one of A and B”, “at least one of A or B”, “A, B or C”, “at least one of A, B and C”, and “at least one of A, B, or C” may each include any one or all the possible combinations of A, B and C. The expression “and/or” is interpreted to include a combination or any of associated elements. For example, the expression “A, B and/or C” may include one of A, B, and C or any combination thereof.

Terms like “first”, “second”, etc., may be simply used to distinguish an element from another, without limiting the elements in a certain sense (e.g., in terms of importance or order). When an element is mentioned as being “coupled” or “connected” to another element with or without an adverb “functionally” or “operatively”, it means that the element may be connected to the other element directly (e.g., wiredly), wirelessly, or through a third element.

It will be further understood that the terms “comprise” and/or “comprising,” when used in this specification, specify the presence of stated features, integers, steps, operations, elements, parts or combinations thereof, but do not preclude the possible presence or addition of one or more other features, integers, steps, operations, elements, components, and/or groups thereof. When an element is mentioned as being “connected to”, “coupled to”, “supported on” or “contacting” another element, it includes not only a case that the elements are directly connected to, coupled to, supported on or contact each other but also a case that the elements are connected to, coupled to, supported on or contact each other through a third element. Throughout the specification, when an element is mentioned as being located “on” another element, it implies not only that the element is abut on the other element but also that a third element exists between the two elements.

A sleep management system according to one or more embodiments will now be described in detail in connection with the accompanying drawings.

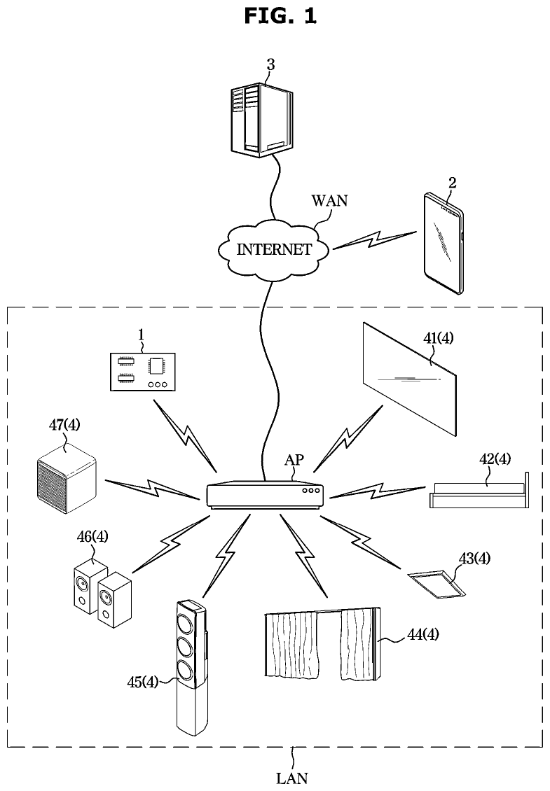

FIG. 1 illustrates an example of a network of a sleep management system, according to an embodiment.

Referring to FIG. 1, the network system according to an embodiment may include a hub device 1, a user device 2, a server device 3 and/or home appliances 4.

The hub device 1 may include a communication module capable of communicating with the user device 2, the server device 3 and/or the home appliances 4, at least one processor for processing data, and at least one memory that stores a program for controlling operation of the hub device 1.

The hub device 1 may obtain processed data by processing data collected from a plurality of sensors. In an embodiment, the hub device 1 may use a machine learning model to process the data collected from the plurality of sensors.

In an embodiment, the hub device 1 may transmit the processed data to the user device 2. For example, the hub device 1 may transmit the processed data to the user device 2 not through the server device 3 but by direct communication.

The home appliances 4 may include various types of electronic products. For example, the home appliances 4 may include at least one of a display device 41, a furniture control device 42, a lighting device 43, an automatic curtain open/close device 44, an air conditioner 45, a speaker 46 and an air purifier 47. The aforementioned home appliances are merely examples, and other various types of electronic products such as a clothes care apparatus in addition to the aforementioned home appliance products may also be included in the home appliances 4.

The home appliance 4 may be controlled remotely by the server device 3.

The furniture control device 42 may include an actuator that may change a posture of the user by changing the structure of the furniture and/or a vibration element that may transmit vibration to the user who lies or sits on the furniture. For example, the furniture control device 42 may include an actuator that is able to control a reclination angle of a recliner bed, a recliner chair and/or a recliner sofa.

The lighting device 43 may include a light source with a controllable intensity and/or color of light.

The automatic curtain open/close device 44 may include an actuator for automatically opening or closing a curtain.

The server device 3 may include a communication module for communicating with the hub device 1, the user device 2 and/or the home appliance 4.

The server device 3 may include at least one processor that may process data received from the hub device 1, the user device 2 and/or the home appliances 4, and at least one memory that may store a program for processing data or processed data. The server device 3 may be implemented with various computing devices such as a workstation, a cloud, a data drive, a data station, etc. The server device 3 may be implemented with one or more servers physically or logically classified based on function, sub-configuration of the function or data, and may transmit or receive data through inter-server communication and process the data.

The server device 3 may perform functions of storing and/or managing a user account, registering the hub device 1, the user device 2 and/or the home appliance 4 by associating them with the user account, and managing or controlling the registered hub device 1 and the home appliance 4. For example, the user may access the server device 3 through the user device 2 to create a user account. The user account may be identified by an identity (ID) and a password created by the user. The user may access the server device 3 through the user device 2 to manage the user account. The server device 3 may register the hub device 1, the user device 2 and/or the home appliance 4 with the user account, according to a set procedure. For example, the server device 3 may connect identification information (e.g., a serial number, a media access control (MAC) address, etc.) of the hub device 1 to the user account to register, manage and control the hub device 1. Likewise, the server device 3 may register the user device 2 and the home appliance 4 with the user account and control them.

The server device 3 may receive various information from the hub device 1, the user device 2 and/or the home appliance 4 registered with the user account.

For example, the server device 3 may include a first server and a second server. The first server may create and/or manage user account information, and register and/or manage information about the hub device 1, the user device 2 and/or the home appliance 4 with the user account. The second server may receive registration information of the user device 2 and the home appliance 4 from the first server to control the user device 2 and/or the home appliance 4.

In another example, the second server may perform a function of managing the hub device 1 and the home appliance 4 registered in the first server on behalf of the first server.

The number of the server devices 3 is not limited thereto, and the server device 3 may include a plurality of servers for performing the same and/or different operations.

The user device 2 may include a communication module for communicating with the hub device 1, the server device 3 and/or the home appliance 4. The user device 2 may include a user interface for receiving user inputs or outputting information for the user. The user device 2 may include at least one processor for controlling operation of the user device 2 and at least one memory for storing a program for controlling the operation of the user device 2.

The user device 2 may be carried by the user or placed at the user's home or office. The user device 2 may include a personal computer, a terminal, a mobile phone, a smart phone, a handheld device, a wearable device, a display device, etc., without being limited thereto.

In the memory of the user device 2, a program, i.e., an application for processing data received from the hub device 1 may be stored. The application may be sold in a state of being installed in the user device 2, or may be downloaded and installed from an external server.

The user may access the server device 3 and create a user account by running the application installed in the user device 2, and register the hub device 1 and/or the home appliance 4 by communicating with the server device 3 based on the login user account.

For example, when the home appliance 4 is operated to access the server device 3 according to a procedure guided in the application installed in the user device 2, the server device 3 may register the home appliance 4 with the user account by registering the identification information (e.g., a serial number or a MAC address) of the home appliance 4 with the user account. The home appliance 4 may also be registered with the user account in the similar manner. It is obvious that other information than the serial number or MAC address of the device to identify the device may be used for the information required to register the device such as the home appliance 4 with the user account.

The user device 2 may receive various information from the hub device 1 and the home appliance 4 registered with the user account directly or through the server device 3.

A network may include both a wired network and a wireless network. The wired network may include a cable network or a telephone network, and the wireless network may include any network that transmits or receives signals in radio waves. The wired network and the wireless network may be connected to each other.

The network may include a wide area network (WAN) such as the Internet, a local area network (LAN) formed around an access point (AP), and a short-range wireless network without an AP. The short-range wireless network may include Bluetooth™ (IEEE 802.15.1), Zigbee (IEEE 802.15.4), wireless fidelity (Wi-Fi) direct, near field communication (NFC), Z-wave, etc., without being limited thereto.

The AP may connect the hub device 1, the user device 2 and/or the home appliance 4 to the WAN connected to the server device 3. The hub device 1, the user device 2 and/or the home appliance 4 may be connected to the server device 3 through the WAN.

The AP may use wireless communication such as Wi-Fi (IEEE 802.11), Bluetooth (IEEE 802.15.1), Zigbee (IEEE 802.15.4), etc., to communicate with the hub device 1, the user device 2 and/or the home appliance 4, and use wired communication to access the WAN, but the wireless communication scheme of the AP is not limited thereto.

In an embodiment, the hub device 1 may communicate with the user device 2 over a short-range wireless network without going through the AP.

For example, the hub device 1 may be connected to the user device 2 over a short-range wireless network (e.g., Wi-Fi direct, Bluetooth™ or NFC). In another example, the hub device 1 may use the long-range wireless network (e.g., a cellular communication module) to be connected to the user device 2 through the WAN.

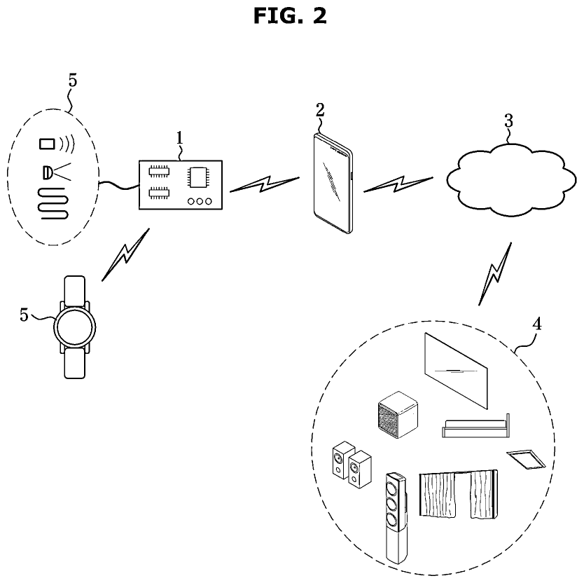

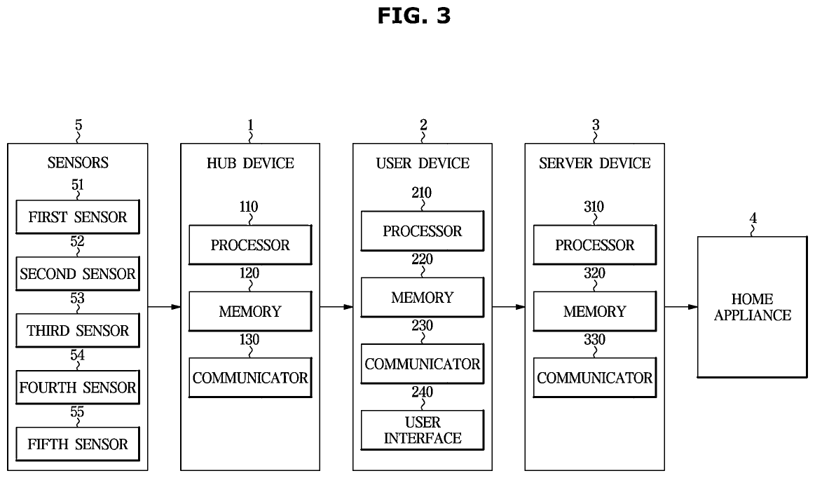

FIG. 2 schematically illustrates an example of a structure of a sleep management system, according to an embodiment. FIG. 3 illustrates an example of a control block diagram of a sleep management system, according to an embodiment.

Referring to FIGS. 2 to 3, the hub device 1 may receive data collected from a plurality of sensors 5.

The plurality of sensors 5 may include sensors (e.g., a first sensor 51, a second sensor 52, a third sensor 53, a fourth sensor 54, and/or a fifth sensor 55) for collecting data of the user.

The plurality of sensors 5 may each collect data of the user and transmit the collected data of the user to the hub device 1.

In an embodiment, the data of the user may include data related to the user's sleep.

The data related to the user's sleep may include pressure data for measuring a pressure change corresponding to a change in posture of the user, displacement data corresponding to displacement of the body that changes according to the user's breathing, oxygen saturation data corresponding to the user's oxygen saturation, electrocardiogram data corresponding to the user's electrocardiogram, acceleration data corresponding to acceleration that changes according to the user's movement, and/or eye-movement data corresponding to the movement of the user's eyes.

The plurality of sensors 5 may include at least two of a pressure sensor for collecting pressure data for measuring a pressure change corresponding to a change in the user's posture, a ultra-wideband (UWB) sensor for measuring displacement data corresponding to displacement of the body that changes according to the user's breathing, an oxygen saturation sensor for collecting oxygen saturation data corresponding to the user's oxygen saturation, an electrocardiogram sensor for collecting electrocardiogram data corresponding to the user's electrocardiogram, an acceleration sensor for collecting acceleration data corresponding to acceleration that changes according to the user's movement, and/or a radar sensor for collecting eye-movement data corresponding to the user's eye movement.

The terms first, second, third, fourth and fifth from the expressions, the first sensor 51, the second sensor 52, the third sensor 53, the fourth sensor 54 and the fifth sensor 55 indicate that the respective sensors are different sensors.

Each of the first sensor 51, the second sensor 52, the third sensor 53, the fourth sensor 54 and the fifth sensor 55 may be one of the pressure sensor, the UWB sensor, the radar sensor, the oxygen saturation sensor, the electrocardiogram sensor or the acceleration sensor.

In the following description, for convenience of explanation, the first sensor 51 is defined as the pressure sensor, the second sensor 52 the UWB sensor, the third sensor 53 the radar sensor, the fourth sensor 54 the oxygen saturation sensor and/or the electrocardiogram sensor, and the fifth sensor 55 the acceleration sensor.

The plurality of sensors 5 may further include an extra sensor (e.g., a mike or camera) in addition to the first to fifth sensors 51 to 55, or may not include at least one of the first to fifth sensors 51 to 55.

Data collected from the plurality of sensors 5 may be sent to the hub device 1.

In an embodiment, data collected by at least one of the plurality of sensors 5 may be sent to the hub device 1 by wired communication, and data collected by the other sensor(s) may be sent to the hub device 1 by wireless communication.

Accordingly, the hub device 1 may be wiredly connected to at least one of the plurality of sensors 5 and wirelessly connected to the other sensor(s).

In an embodiment, the fourth sensor 54 may be included in a smart sensor device (e.g., a wearable device). The smart sensor device may include a wireless communication module and the fourth sensor 54. For example, the smart sensor device may include a smart watch that is shaped like a watch and/or a smart ring that is shaped like a ring, but the form of the smart sensor device is not limited thereto.

The smart sensor device may establish wireless communication with the hub device 1, and transmit data collected from the fourth sensor 54 to the hub device 1 by wireless communication.

In an embodiment, the smart sensor device may include the fourth sensor 54 and the fifth sensor 55. The smart sensor device may establish wireless communication with the hub device 1, and transmit data collected from the fourth sensor 54 and the fifth sensor 55 to the hub device 1 by wireless communication.

In an embodiment, the data collected from the plurality of sensors 5 may be sent to the hub device 1 by wired communication.

In one or more embodiments, the plurality of sensors 5 may include an imaging sensor (e.g., a camera). However, in an embodiment, the plurality of sensors 5 may all be non-imaging sensors.

In an embodiment of the disclosure, as all the plurality of sensors 5 correspond to non-imaging sensors, an invasion of the user's privacy may be prevented.

In an embodiment, the hub device 1 may include at least one memory 120 for storing a program for processing data collected from the plurality of sensors 5, and at least one processor 110 that is able to process the data collected from the plurality of sensors 5 based on the program stored in the at least one memory 120.

The at least one memory 120 may store a machine learning model for processing the data collected from the plurality of sensors 5.

In an embodiment, the machine learning model may be one for feature extraction, which extracts a feature of the data collected from the plurality of sensors 5 when the data is input thereto, and outputs processed data including the extracted feature.

The feature of the data may include elements extracted from the data by the machine learning model to perform classification or prediction.

In an embodiment, the machine learning model may be one for sleep stage decision, which outputs processed data including data of a sleep stage of the user when the data collected from the plurality of sensors 5 is input thereto.

For example, the at least one memory 120 may include a first machine learning model 11 for processing first data collected from the first sensor 51, a second machine learning model 12 for processing second data collected from the second sensor 52, a third machine learning model 13 for processing third data collected from the third sensor 53, a fourth machine learning model 14 for processing fourth data collected from the fourth sensor 54, and a fifth machine learning model 15 for processing fifth data collected from the fifth sensor 55.

The at least one processor 110 may obtain first processed data by processing the first data collected from the first sensor 51.

The first processed data may include feature data extracted from the first data and/or data about a sleep stage extracted from the first data. The volume of the first processed data may be smaller than the volume of the first data.

The at least one processor 110 may obtain second processed data by processing the second data collected from the second sensor 52.

The second processed data may include feature data extracted from the second data and/or data about a sleep stage extracted from the second data. The volume of the second processed data may be smaller than the volume of the second data.

The at least one processor 110 may obtain third processed data by processing the third data collected from the third sensor 53.

The third processed data may include feature data extracted from the third data and/or data about a sleep stage extracted from the third data. The volume of the third processed data may be smaller than the volume of the third data.

The at least one processor 110 may obtain fourth processed data by processing the fourth data collected from the fourth sensor 54.

The fourth processed data may include feature data extracted from the fourth data and/or data about a sleep stage extracted from the fourth data. The volume of the fourth processed data may be smaller than the volume of the fourth data.

The at least one processor 110 may obtain fifth processed data by processing the fifth data collected from the fifth sensor 55.

The fifth processed data may include feature data extracted from the fifth data and/or data about a sleep stage extracted from the fifth data. The volume of the fifth processed data may be smaller than the volume of the fifth data.

In the disclosure, the hub device 1 (primarily) processes data and transmits it to the user device 2, thereby reducing data throughput to be borne by the user device 2.

The hub device 1 may include a communicator 130 including a wired communication module for performing wired communication with the plurality of sensors 5, and/or a wireless communication module for performing wireless communication with the user device 2, the server device 3 and/or the home appliances.

The hub device 1 may include a printed circuit board (PCB) including the at least one processor 110, the at least one memory 120 and the communicator 130. At least some of the plurality of sensors 5 may be wiredly connected to the PCB.

The hub device 1 may include a housing that covers the PCB.

The hub device 1 is likely to be installed in a location where user operation is difficult. Hence, in an embodiment, the hub device 1 may not include any user interface device (input/output device).

In preparation for an occasion when the hub device 1 is installed in a place where user may easily operate the hub device 1, the hub device 1 may include a user interface device (input/output device) in an embodiment.

In an embodiment, the user may operate the user interface device configured in the hub device 1 to connect the hub device 1 to an AP.

In an embodiment, the user may operate the user interface device configured in the hub device 1 to activate the communicator 130 of the hub device 1.

In an embodiment, the user may operate the user interface device configured in the hub device 1 to power on the hub device 1.

The at least one processor 110 may control the plurality of sensors 5.

For example, the at least one processor 110 may control at least one of the plurality of sensors 5 connected via wires.

The at least one processor 110 may wake up at least one of the plurality of sensors 5 connected via wires based on a sensor wakeup condition being satisfied.

The waking up of the sensor may include activating the sensor.

The at least one processor 110 may switch at least one of the plurality of sensors 5 connected via wires into a standby state based on a sensor standby condition being satisfied. The switching of the sensor into the standby state may include inactivating the sensor or driving the sensor in a low power mode.

The user device 2 may receive, from the hub device 1, data obtained by the hub device 1. In an embodiment, the user device 2 may receive, from the hub device 1, data obtained by the hub device 1 by wireless communication. In one or more embodiments, the hub device 1 and the user device 2 may be wiredly connected via a connection cable. The user device 2 may receive, from the hub device 1, data obtained by the hub device 1 by wired communication.

The data obtained by the hub device 1 may include processed data resulting from processing of the data collected from the plurality of sensors 5.

In an embodiment, the user device 2 may include at least one memory 220 for storing a program for processing data received from the hub device 1, and at least one processor 210 that is able to process the data received from the hub device 1 based on the program stored in the at least one memory 220.

The at least one memory 220 may store a machine learning model for processing the data received from the hub device 1.

The at least one memory 220 may store a sleep management application that is downloadable from an external server. The sleep management application may be a downloadable app, at least a portion of which may be at least temporarily stored or arbitrarily created in a recording medium that may be readable to a device such as a server of the manufacturer, a server of the application store, or a relay server.

The sleep management application may include a machine learning model. The machine learning model included in the sleep management application may be updated by the external server.

In an embodiment, the user device 2 may include a communicator 230 including at least one communication module for establishing communication with the hub device 1, the server device 3, the home appliance 4 and/or the smart sensor device including at least some of the plurality of sensors 5.

The user device 2 may receive the processed data from the hub device 1 through the communicator 230.

In an embodiment, the at least one processor 210 may establish communication between the communicator 130 of the hub device 1 and the communicator 230 (e.g., a short-range wireless communication module) of the user device 2 in response to the communicator 230 being activated.

In an embodiment, the at least one processor 210 may control a user interface 240 to provide feedback that requests activation of the communicator 230 in response to the sleep management application being executed while the communicator 230 (e.g., the short-range wireless communication module) is not activated.

The at least one processor 210 may wirelessly receive data from the hub device 1 through the communicator 230 (e.g., the short-range wireless communication module).

The at least one processor 210 may use the machine learning model stored in the at least one memory 220 to process the data received from the hub device 1.

The data received from the hub device 1 may include the first processed data, the second processed data, the third processed data, the fourth processed data and/or the fifth processed data.

The at least one processor 210 may obtain sleep state information associated with a sleep state of the user by inputting the first processed data, the second processed data, the third processed data, the fourth processed data and/or the fifth processed data to the machine learning model stored in the at least one memory 220.

The sleep state information associated with the user's sleep state may include at least one of information about the user's sleep stage, information about the user's stress index, or information about the user's sleep disorder.

The machine learning model stored in the at least one memory 220 may be one for decision of the user's sleep state, which outputs sleep state information associated with the user's sleep state when the first processed data, the second processed data, the third processed data, the fourth processed data and/or the fifth processed data are input thereto.

The at least one processor 210 may store the sleep state information obtained by inputting the first processed data, the second processed data, the third processed data, the fourth processed data and/or the fifth processed data to the machine learning model in the at least one memory 220.

In an embodiment, the at least one processor 210 may generate sleep summary information based on the sleep state information accumulated and stored in the at least one memory 220. The at least one processor 210 may control the user interface 240 to output sensory information corresponding to the sleep summary information based on a preset condition being satisfied (e.g., based on receiving of a user input to check the sleep summary information).

The at least one processor 210 may control the communicator 230 to transmit the sleep state information obtained by inputting the first processed data, the second processed data, the third processed data, the fourth processed data and/or the fifth processed data to the machine learning model.

The communicator 230 may include a first communication module for establishing communication with the hub device 1 and a second communication module for establishing communication with the server device 3. Accordingly, the communicator 230 may communicate with the hub device 1 in a first communication scheme and at the same time, communicate with the server device 3 in a second communication scheme.

In an embodiment of the disclosure, the sleep state information may be obtained after the data collected from the plurality of sensors 5 is (primarily) processed by the hub device 1 and (secondarily) processed by the user device 2, and the sleep state information may be transmitted to the server device 3 in real time.

In an embodiment of the disclosure, the data collected from the plurality of sensors 5 and associated with the user's privacy may not be sent directly to the server device 3.

The user device 2 may include the user interface 240 for communication with the user.

In one or more embodiments, it is also possible to obtain the sleep state information after the hub device 1 (primarily) processes the data collected from the plurality of sensors 5 and (secondarily) processes the (primarily) processed data.

In one or more embodiments, it is also possible to obtain the sleep state information after the hub device 1 (primarily) processes the data collected from the plurality of sensors 5 and transmits the (primarily) processed data to the server device 3, and the server (secondarily) processes the (primarily) processed data.

In one or more embodiments, it is also possible to obtain the sleep state information after the hub device 1 transmits the data collected from the plurality of sensors 5 to the user device 2, and the user device 2 (primarily) processes the data received from the hub device 1 and (secondarily) processes the (primarily) processed data.

In one or more embodiments, it is also possible to obtain the sleep state information after the hub device 1 transmits the data collected from the plurality of sensors 5 to the server device 3 and the server device 3 (primarily) processes the data received from the hub device 1 and (secondarily) processes the (primarily) processed data.

The user interface 240 may obtain a user input. The user interface 240 may provide various information about operations of the user device 2. The user interface 240 may include an input interface and an output interface.

The input interface may convert the sensory information received from the user into an electric signal. The electric signal may correspond to a user input. The user input may include various commands. The input interface may transmit the electric signal (voltage or current) corresponding to the user input to the at least one processor 210.

The input interface may include various input devices to convert tactile information to an electric signal. For example, the input interface may be configured with a physical button or a touch screen. The input interface may include a mike to convert auditory information to an electric signal.

The input interface may receive a user input to run the sleep management application.

The output interface may output information associated with operations of the user device 2. The output interface may display information input by the user or information to be provided for the user in various screens. The output interface may display information regarding an operation of the user device 2 in at least one of an image or text. For example, the output interface may output an interface of the sleep management application. Furthermore, the output interface may display a graphic user interface (GUI) that enables the user device 2 to be controlled. In other words, the output interface may display a user interface element (UI element) such as an icon.

The output interface may output an interface corresponding to the sleep management application.

For example, the output interface may include a liquid crystal display (LCD) panel, a light emitting diode (LED) panel, an organic LED (OLED) panel, or a micro LED panel. The output interface may include a touch display that serves as an input device as well.

The output interface and the input interface may be configured separately or in one device (e.g., the touch display).

The server device 3 may receive, from the user device 2, data obtained by the user device 2 by wireless communication.

The data obtained by the user device 2 may include the aforementioned sleep state information.

In an embodiment, the server device 3 may include at least one memory 320 for storing a program for processing the sleep state information received from the user device 2, and at least one processor 310 that is able to process the sleep state information received from the user device 2 based on the program stored in the at least one memory 320.

The at least one memory 320 may store the sleep state information received from the user device 2.

The at least one memory 320 may store a program for generating sleep summary information based on the sleep state information received from the user device 2.

The at least one processor 310 may generate the sleep summary information based on the sleep state information accumulated and stored in the at least one memory 320.

The at least one memory 320 may store a program for controlling the home appliance 4 based on the sleep state information received from the user device 2.

The at least one processor 310 may control the home appliance 4 based on the sleep state information received from the user device 2.

In one or more embodiments, the program for generating the sleep summary information based on the sleep state information and the program for controlling the home appliance 4 based on the sleep state information may be stored in different servers.

For example, a first server included in the server device 3 may store the program for generating the sleep summary information based on the sleep state information, and a second server included in the server device 3 may store the program for controlling the home appliance 4 based on the sleep state information.

The at least one memory 320 may store the sleep summary information based on the sleep state information.

In an embodiment, the server device 3 may include a communicator 330 including at least one communication module for establishing communication with the hub device 1, the user device 2, the home appliance 4 and/or the smart sensor device including at least some of the plurality of sensors 5.

The server device 3 may receive the sleep state information from the user device 2 through the communicator 330.

The server device 3 may transmit a control command to the home appliance 4 through the communicator 330.

The server device 3 may transmit the sleep summary information to the user device 2 through the communicator 330.

In an embodiment of the disclosure, as the server device 3 performs various operations based on the sleep state information, the user's sleep may be managed in various ways.

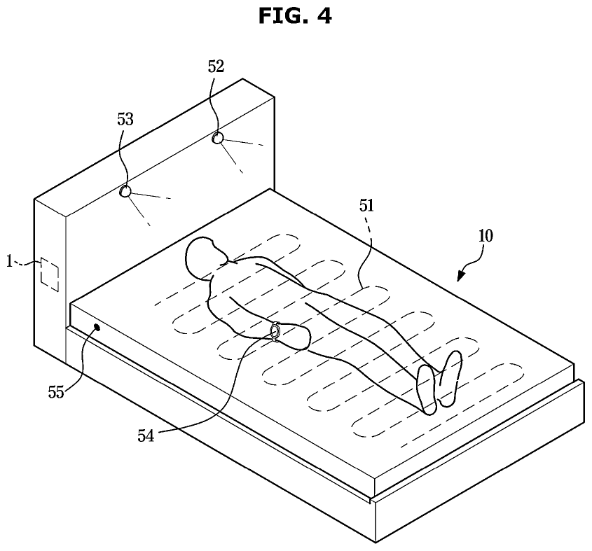

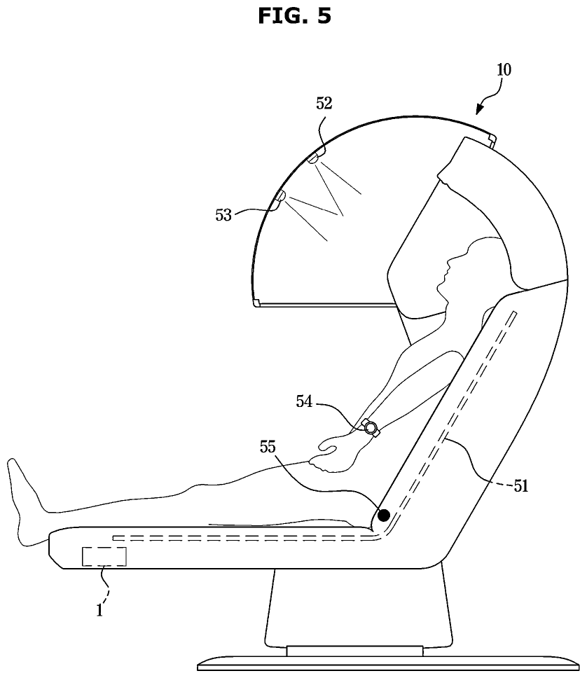

FIGS. 4 and 5 illustrate an example of a plurality of sensors of a sleep management system, according to an embodiment.

Referring to FIGS. 4 and 5, in an embodiment, some of the plurality of sensors 5 (e.g., the first sensor 51, the second sensor 52 and/or the third sensor 53) may be installed on a piece of furniture 10, and the others (e.g., the fourth sensor 54 and/or the fifth sensor 55) among the plurality of sensors 5 may be configured in the smart sensor device (e.g., a smart watch, a smart ring, etc.).

At least some of the plurality of sensors 5 may be configured on the furniture 10 on which the user may sit or lie.

The furniture 10 on which the user may sit or lie may include, for example, a bed, a chair and/or a sofa, but obviously, any furniture having the form that allows the user to sit or lie thereon may be used as the furniture 10 without limitation.

In an embodiment, the furniture 10 such as a bed, a chair and/or a sofa may include an actuator that is able to change the user's posture by changing its structure and/or a vibration element capable of transmitting vibration to the user.

The first sensor 51 may include a pressure sensor. The pressure sensor may include a piezoelectric element that generates an electric signal corresponding to displacement created by the pressure.

The first sensor 51 may be installed in a location where the pressure created by the user's body (e.g., the whole body) when the user lies or sits may be measured.

For example, when the furniture 10 corresponds to a bed, the first sensor 51 may be configured in a mattress where pressure occurs by the user's body. The mattress may include a cover having a polygonal or circular flat shape, defining the exterior and having an accommodation space, and a pad arranged in the accommodation space of the cover and including the first sensor 51. The mattress may be placed on the floor, a chair, a sofa or a bed.

The mattress may further include springs and/or sponge. The spring and/or the sponge may be arranged in the accommodation space of the cover.

The structure (e.g., length, layout, etc.) of the first sensor 51 may vary by the size of the mattress.

In another example, when the furniture 10 corresponds to a chair, the first sensor 51 may be configured in a seating portion, a backrest portion, a headrest and/or a leg portion where pressure occurs by the user's body.

The seating portion may include a portion coming into contact with the user's buttocks, the backrest portion may include a portion coming into contact with the user's back, the headrest may include a portion coming into contact with the user's head, and the leg portion may include a portion coming into contact with the user's legs.

The location of the first sensor 51 is not limited to the example shown in FIGS. 4 and 5, and the first sensor 51 may be installed at various locations where the pressure created by the body of the user who lies or sits on the furniture 10 may be measured.

The first sensor 51 may measure the pressure created by the user who lies or sits on the furniture 10. For example, the first sensor 51 may measure a distribution of the pressure that occurs by the user who lies or sits on the furniture 10. The first sensor 51 may obtain pressure data corresponding to the pressure created by the user who lies or sits on the furniture 10.

The second sensor 52 may include a UWB sensor. The UWB sensor may include a UWB signal irradiator for transmitting an ultra-wide band (UWB) signal and a UWB signal receiver for receiving a UWB signal reflected by the user's body.

The second sensor 52 may have a detection region facing the body (e.g., torso) of the user who lies or sits on the furniture 10. The second sensor 52 may have a detection region that may detect displacement of the body caused by the user's breathing. The second sensor 52 may be configured on the frame of the furniture 10 to have the detection region facing the body (e.g., torso) of the user, but the location of the second sensor 52 is not limited thereto.

For example, the second sensor 52 may have a detection region facing a portion of the body of the user who lies or sits on the furniture 10.

For example, when the furniture 10 corresponds to a bed, the second sensor 52 may have a detection region facing a center portion of the bed.

In another example, when the furniture 10 corresponds to a chair, the second sensor 52 may have a detection region facing a backrest portion of the chair.

The second sensor 52 may transmit a UWB signal to the body of the user and receive a UWB signal reflected from the body of the user.

The second sensor 52 may measure displacement of the user's body based on the UWB signal reflected from the body of the user. For example, the second sensor 52 may measure displacement of the user's body based on a time of flight (ToF) of the UWB signal. In another example, the second sensor 52 may use the Doppler effect to measure the displacement of the user's body according to a change in wavelength (and frequency) of the UWB signal.

In other words, the second sensor 52 may obtain displacement data corresponding to the displacement of the body that changes according to the user's breathing.

The third sensor 53 may include a radar sensor. The third sensor 53 may include a radar signal irradiator for transmitting a radar signal (e.g., millimeter waves or an mmWave signal) and a radar signal receiver for receiving a radar signal (e.g., an mmWave signal) reflected from the user's body.

A frequency band (e.g., 28 GHz) of the radar signal output from the third sensor 53 may be higher than the frequency band (e.g., 6.0 to 8.8 GHz) of the UWB signal output from the second sensor 52.

A bandwidth of the radar signal output from the third sensor 53 may be narrower than the bandwidth of the UWB signal output from the second sensor 52.

The third sensor 53 may have a detection region facing the body (e.g., face) of the user who lies or sits on the furniture 10. The third sensor 53 may have a detection region that may detect a movement of eyes of the user. The third sensor 53 may be configured on the frame of the furniture 10 to have the detection region facing the user's body (e.g., torso), but the position of the third sensor 53 is not limited thereto.

The third sensor 53 may have a detection region facing a portion of the body of the user who lies or sits on the furniture 10.

For example, when the furniture 10 corresponds to a bed, the third sensor 53 may have a detection region facing a head area of the bed.

In another example, when the furniture 10 corresponds to a chair, the third sensor 53 may have a detection region facing the headrest of the chair.

The third sensor 53 may transmit a radar signal (mmWave signal) to the body of the user and receive an mmWave signal reflected from the body of the user.

The third sensor 53 may measure a movement of the eyes of the user based on the mmWave reflected from the eyes of the user.

In other words, the third sensor 53 may obtain eye-movement data corresponding to the movement of the eyes of the user.

The fourth sensor 54 may include an oxygen saturation sensor and/or an electrocardiogram sensor. The oxygen saturation sensor and/or the electrocardiogram sensor may include a light source for irradiating light and a photo receiver for receiving light reflected from the user's body.

The fourth sensor may have a detection region facing the body of the user who lies or sits on the furniture 10.

The fourth sensor 54 may be arranged in a smart sensor device (e.g., a smart watch, a smart ring, etc.) that may be worn by the user.

The fourth sensor 54 may operate in a non-invasive manner, irradiating light to a portion (e.g., a wrist) of the user's body and receiving light reflected from the user's body.

The fourth sensor 54 may measure an oxygen saturation level in the user's blood and an electrocardiogram (ECG) of the user based on the intensity of the light reflected from the user's body.

A portion of the light irradiated to a portion of the body may be absorbed in a blood vessel, and the oxygen saturation level in the user's blood or the user's ECG may be measured according to the light absorption rate and patterns of the absorbed light.

The fifth sensor 55 may include an acceleration sensor. The acceleration sensor may include a microelectromechanical system (MEMS) sensor, a 3-axis acceleration sensor and/or a 6-axis acceleration sensor.

In one or more embodiments, like the fourth sensor 54, the fifth sensor 55 may be configured in a smart sensor device (e.g., a smart watch, a smart ring, etc.) that may be worn by the user.

In an embodiment, the fifth sensor 55 may be installed on the furniture 10.

The fifth sensor 55 may obtain acceleration data corresponding to the movement of the user's body.

The hub device 1 may be installed on the furniture 10 or at a location adjacent to the furniture 10 and wiredly connected to some of the plurality of sensors 5. Furthermore, the hub device 1 may perform wireless communication with the smart sensor device (e.g., a wearable device) worn by the user.

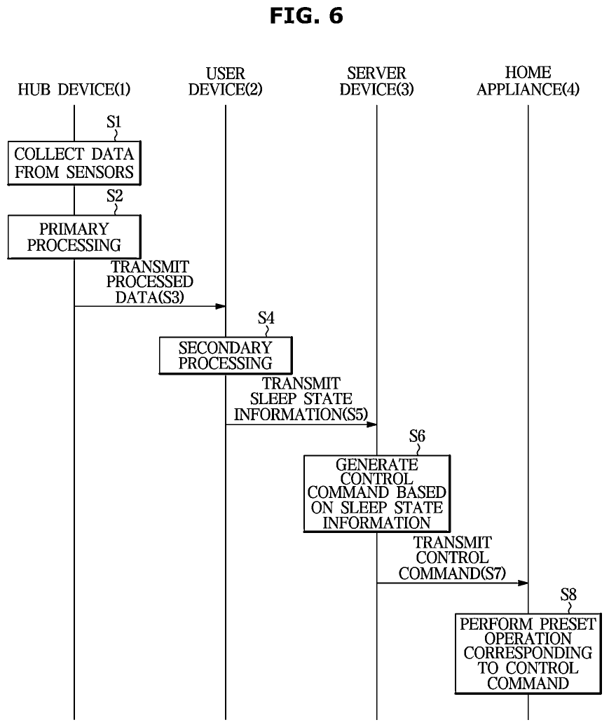

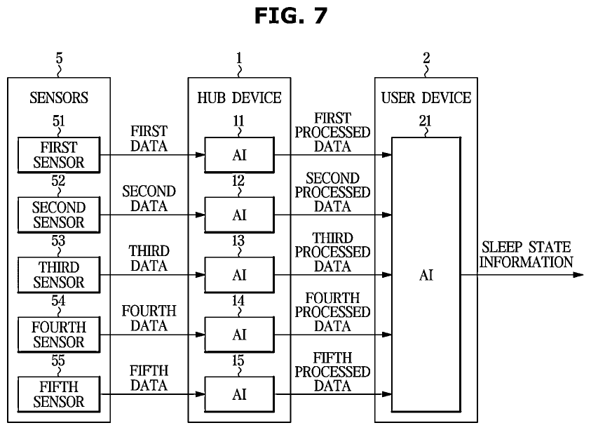

FIG. 6 illustrates an example of a flowchart of a sleep management system, according to an embodiment. FIG. 7 is a diagram for describing a procedure for processing data collected from a plurality of sensors of a sleep management system, according to an embodiment.

Referring to FIGS. 6 to 7, the hub device 1 may collect data from the plurality of sensors 5, in S1.

When the plurality of sensors 5 maintain in an active state, there may be a lot of consumption of power supplied to the plurality of sensors 5. The plurality of sensors 5 maintaining in the active state may include the plurality of sensor 5 maintaining in a state of obtaining sensor data by receiving power.

In an embodiment, the hub device 1 may maintain at least some of the plurality of sensors 5 in an inactive state and change the plurality of sensors 5 into an active state based on a preset condition being satisfied.

For example, the hub device 1 may determine whether there is a user on the furniture 10 by processing the data collected from the plurality of sensors 5. The presence of a user on the furniture 10 may include the user lying or sitting on the furniture 10.

The hub device 1 may switch the plurality of sensors 5 into a standby state based on determining that there is no user on the furniture 10.

For example, the hub device 1 may deactivate other sensors than the first sensor 51 and operate the first sensor 51 in a low power mode. The deactivating of the sensor may include blocking power supplied to the sensor.

The operating of the sensor in the low power mode may include setting an operation period (e.g., a data collection period) of the sensor to be longer.

The hub device 1 may determine whether there is the user on the furniture 10 by processing the data collected by the sensor (e.g., the first sensor 51) operating in the low power mode among the plurality of sensors 5.

The hub device 1 may wake up the plurality of sensors 5 based on determining that there is a user on the furniture 10.

Based on activating of the plurality of sensors 5, the collected data may be transmitted to the hub device 1.

The first sensor 51 may send the first data to the hub device 1, the second sensor 52 may send the second data to the hub device 1, the third sensor 53 may send the third data to the hub device 1, the fourth sensor 54 may send the fourth data to the hub device 1, and the fifth sensor 55 may send the fifth data to the hub device 1.

In an embodiment, at least some of the plurality of sensors 5 may send the sensor data to the hub device 1 by wired communication, and the others of the plurality of sensors 5 may send the sensor data to the hub device 1 by wireless communication.

The hub device 1 may (primarily) process the data collected from the plurality of sensors 5, in S2. For this, the hub device 1 may be equipped with a machine learning model.

The data collected from the plurality of sensors 5 may correspond to raw data, which has a large volume or include data related to the user's privacy. Hence, the hub device 1 according to an embodiment of the disclosure may (primarily) process the raw data collected from the plurality of sensors 5. In order for the user device 2 to process all the raw data collected from the plurality of sensors 5, the user device 2 needs to be available for communication with all the plurality of sensors 5, but there is a limitation on the number of communication modules in the user device 2, so the separate hub device 1 may be required to receive and process the data.

The data collected from the plurality of sensors 5 may be processed by at least one of the machine learning models (the first machine learning model 11, the second machine learning model 12, the third machine learning model 13, the fourth machine learning model 14, or the fifth machine learning model 15) installed in the hub device 1.

The first machine learning model 11 for extracting a feature from the first data collected from the first sensor 51, a second machine learning model 12 for extracting a feature from the second data collected from the second sensor 52, a third machine learning model 13 for extracting a feature from the third data collected from the third sensor 53, a fourth machine learning model 14 for extracting a feature from the fourth data collected from the fourth sensor 54, and a fifth machine learning model 15 for extracting a feature from the fifth data collected from the fifth sensor 55 may be installed in the hub device 1.

The first machine learning model 11 may be pre-trained to extract a feature from pressure data collected by the pressure sensor. The second machine learning model 12 may be pre-trained to extract a feature from displacement data collected by the UWB sensor. The third machine learning model 13 may be pre-trained to extract a feature from eye-movement data collected by the radar sensor. The fourth machine learning model 14 may be pre-trained to extract a feature from oxygen saturation data and/or ECG data collected by the oxygen saturation sensor and/or the ECG sensor. The fifth machine learning model 15 may be pre-trained to extract a feature from acceleration data collected by the acceleration sensor.

The first machine learning model 11 may use the first data collected by the first sensor 51 as input data to output the first processed data as output data. The first processed data may include, for example, information about the user's movement, posture, respiration rate and heart rate inferred by the first data. In another example, the first processed data may include information about the user's sleep stage, information about the user's stress index, or information about the user's sleep disorder inferred by the first data.

The second machine learning model 12 may use the second data collected by the second sensor 52 as input data to output the second processed data as output data. The second processed data may include, for example, information about a respiration rate and a heart rate inferred by the second data. In another example, the second processed data may include information about the user's sleep stage, information about the user's stress index, or information about the user's sleep disorder inferred by the second data.

The third machine learning model 13 may use the third data collected by the third sensor 53 as input data to output the third processed data as output data. The third processed data may include, for example, information about an eye movement inferred by the third data. In another example, the third processed data may include information about the user's sleep stage, information about the user's stress index, or information about the user's sleep disorder inferred by the third data.

The fourth machine learning model 14 may use the fourth data collected by the fourth sensor 54 as input data to output the fourth processed data as output data. The fourth processed data may include, for example, information about an oxygen saturation level and/or an ECG inferred by the fourth data. In another example, the fourth processed data may include information about the user's sleep stage, information about the user's stress index, or information about the user's sleep disorder inferred by the fourth data.

The fifth machine learning model 15 may use the fifth data collected by the fifth sensor 55 as input data to output the fifth processed data as output data. The fifth processed data may include, for example, information about a movement inferred by the fifth data. In another example, the fifth processed data may include information about the user's sleep stage, information about the user's stress index, or information about the user's sleep disorder inferred by the fifth data.

In the disclosure, the hub device 1 may (primarily) process the data collected from the plurality of sensors 5 so that the data collected from the plurality of sensors 5 may be sent to the user device 2 in a communication scheme with low data transfer capacity.

In the disclosure, by (primarily) processing the UWB data with a high volume of raw data, data throughput to be borne by the processor 210 of the user device 2 may be reduced.

The hub device 1 may process the data collected from the plurality of sensors 5, and transmit the processed data to the user device 2, in S3.

In the disclosure, the user device 2 may consequently process the data collected from the plurality of sensors 5 even when equipped only with the communication module capable of communicating with the hub device 1.

The hub device 1 may transmit the processed data to the user device 2 by wireless communication. In an embodiment, the communicator 130 of the hub device 1 may include a first communication module for receiving data collected from some (e.g., the fourth sensor 54) of the plurality of sensors 5 in a first wireless communication scheme, and a second communication module for transmitting the processed data to the user device 2 in a second wireless communication scheme.

The first wireless communication scheme and the second wireless communication scheme may be the same or different from each other.

In an embodiment of the disclosure, as the hub device 1 is equipped with both the first communication module for receiving data from some of the plurality of sensors 5 and the second communication module for communicating with the user device 2, the hub device 1 may be able to communicate with the plurality of sensors 5 and the user device 2 at the same time.

The user device 2 may process the processed data received from the hub device 1, in S4. For this, the user device 2 may be equipped with a machine learning model.

The processed data received from the hub device 1 may be processed by a machine learning model 21 installed in the user device 2.

The machine learning model 21 installed in the user device 2 may include an artificial neural network (deep neural network) model with several layers (e.g., an input layer, a hidden layer, and an output layer). The machine learning model 21 installed in the user device 2 may be configured in a perceptron structure that receives multiple signals and outputs one signal. The machine learning model 21 installed in the user device 2 may be trained for a purpose of estimating the user's sleep state based on the processed data processed by the hub device 1.

The machine learning model 21 installed in the user device 2 may use the processed data output by the machine learning model of the hub device 1 as input data to output sleep state information associated with the user's sleep state as output data.

The machine learning model 21 equipped in the user device 2 may use the first processed data, the second processed data, the third processed data, the fourth processed data and/or the fifth processed data as input data to output the sleep state information.

The first processed data, the second processed data, the third processed data, the fourth processed data and/or the fifth processed data may include probability information of the user's sleep stage.

For example, the first processed data, the second processed data, the third processed data, the fourth processed data and/or the fifth processed data may include probability data of the user's sleep stage corresponding to an awakening stage, a REM sleep stage, and an N1 stage, an N2 stage, an N3 stage or an N4 stage of a non-REM sleep stage.

The first processed data may include a probability value a1 of the user's sleep stage being the awakening stage, a probability value b1 of being REM sleep stage, a probability value c1 of being N1 stage of the non-REM sleep stage, a probability value d1 of being N2 stage of the non-REM sleep stage, a probability value e1 of being N3 stage of the non-REM sleep stage and a probability value f1 of being N4 stage of the non-REM sleep stage.

The second processed data may include a probability value a2 of the user's sleep stage being the awakening stage, a probability value b2 of being REM sleep stage, a probability value c2 of being N1 stage of the non-REM sleep stage, a probability value d2 of being N2 stage of the non-REM sleep stage, a probability value e2 of being N3 stage of the non-REM sleep stage and a probability value f2 of being N4 stage of the non-REM sleep stage.

The third processed data may include a probability value a3 of the user's sleep stage being the awakening stage, a probability value b3 of being REM sleep stage, a probability value c3 of being N1 stage of the non-REM sleep stage, a probability value d3 of being N2 stage of the non-REM sleep stage, a probability value e3 of being N3 stage of the non-REM sleep stage and a probability value f3 of being N4 stage of the non-REM sleep stage.

The fourth processed data may include a probability value a4 of the user's sleep stage being the awakening stage, a probability value b4 of being REM sleep stage, a probability value c4 of being N1 stage of the non-REM sleep stage, a probability value d4 of being N2 stage of the non-REM sleep stage, a probability value e4 of being N3 stage of the non-REM sleep stage and a probability value f4 of being N4 stage of the non-REM sleep stage.

The fifth processed data may include a probability value a5 of the user's sleep stage being the awakening stage, a probability value b5 of being REM sleep stage, a probability value c5 of being N1 stage of the non-REM sleep stage, a probability value d5 of being N2 stage of the non-REM sleep stage, a probability value e5 of being N3 stage of the non-REM sleep stage and a probability value f5 of being N4 stage of the non-REM sleep stage.

The machine learning model 21 installed in the user device 2 may finally determine the user's sleep stage by assigning different weights to the respective probability values (a1, a2, a3, a4, a5, b1, b2, b3, b4, b5, c1, c2, c3, c4, c5, d1, d2, d3, d4, d5, e1, e2, e3, e4, e5, f1, f2, f3, f4, f5) included in the first to fifth processed data.

For example, as the user's eye movement is important in determining whether the user's sleep stage is the REM sleep stage, the machine learning model 21 may give the highest weight to the probability value b3 included in the third processed data among the probability values b1, b2, b3, b4 and b5 in determining whether the user's sleep stage is the REM sleep stage.

In another example, as the user's body movement is important in determining whether the user's sleep stage is the N4 stage, the highest weights may be given to the probability value f1 included in the first processed data and the probability value f5 included in the fifth processed data among f1, f2, f3, f4 and f5 in determining whether the user's sleep stage is the N4 stage.

The first processed data, the second processed data, the third processed data, the fourth processed data and/or the fifth processed data may include the user's stress index values.

For example, the first processed data may include the user's stress index value g1, the second processed data the user's stress index value g2, the third processed data the user's stress index value g3, the fourth processed data the user's stress index value g4, and the fifth processed data the user's stress index g5.

The machine learning model 21 installed in the user device 2 may finally determine the user's stress index by assigning different weights to the respective stress index values included in the first to fifth processed data.

For example, as the user's heart rate or ECG is important in determining the user's stress index, the machine learning model 21 may give the highest weight to the stress index value g2 included in the second processed data and the stress index value g4 included in the fourth processed data among the stress index values g1, g2, g3, g4 and g5 in determining the user's stress index.

The first processed data, the second processed data, the third processed data, the fourth processed data and/or the fifth processed data may include probability values of the user's sleep disorder.

For example, the first processed data may include the user's snoring probability value h1, sleepwalking probability value i1 and/or apnea-hypopnea index j1, the second processed data may include the user's snoring probability value h2, sleepwalking probability value i2, and/or apnea-hypopnea index j2, the third processed data may include the user's snoring probability value h3, sleepwalking probability value i3, and/or apnea-hypopnea index j3, the fourth processed data may include the user's snoring probability value h4, sleepwalking probability value i4, and/or apnea-hypopnea index j4, and the fifth processed data may include the user's snoring probability value h5, sleepwalking probability value i5, and/or apnea-hypopnea index j5.

The machine learning model 21 installed in the user device 2 may finally determine whether snoring of the user appears by assigning different weights to the respective snoring probability values (h1, h2, h3, h4 and h5) included in the first to fifth processed data.

The machine learning model 21 installed in the user device 2 may finally determine whether sleepwalking of the user appears by assigning different weights to the respective sleepwalking probability values (i1, i2, i3, i4 and i5) included in the first to fifth processed data.

The machine learning model 21 installed in the user device 2 may finally determine the user's apnea-hypopnea index by assigning different weights to the respective apnea-hypopnea indexes (j1, j2, j3, j4 and j5) included in the first to fifth processed data.

The sleep state information output by the machine learning model 21 installed in the user device 2 may include at least one of information about the user's sleep stage, information about the user's stress index, or information about the user's sleep disorder.

In the disclosure, as the data (primarily) output by at least one of the machine learning models (the first machine learning model 11, the second machine learning model 12, the third machine learning model 13, the fourth machine learning model 14, or the fifth machine learning model 15) installed in the hub device 1 is (secondarily) input to the machine learning model 21 installed in the user device 2 to output the sleep state information, the user's sleep state may be accurately estimated. In other words, in the disclosure, a large amount of data may be processed in stages, thereby accurately estimating the user's sleep state.

The user device 2 may transmit the sleep state information to the server device 3, in S5.

In one or more embodiments, the user device 2 may generate a control command to control the home appliance 4 based on the sleep state information, and transmit the control command to control the home appliance 4 to the server. In other words, operation S6, which will be described below, may be performed by the user device 2.

In an embodiment, the user device 2 may generate the control command to control the home appliance 4 based on the sleep state information and transmit the control command to the server device 3, and the server device 3 may forward the control command received from the user device 2 to the home appliance 4.

In the disclosure, instead of transmitting data directly involved with the user's privacy to the server device 3, only the sleep state information associated with the user's sleep state may be transmitted to the server device 3, thereby gaining the user's agreement on the data collection more easily.

The server device 3 may generate the control command to control the home appliance 4 based on the sleep state information, in S6. In this case, the home appliance 4 may include at least one home appliance 4 connected and registered with the user device 2. The server device 3 may store and/or manage the user account, and register the user device 2 and the home appliance 4 by associating them with the user account.

The at least one home appliance 4 connected and registered with the user device 2 may include the home appliance 4 registered with the user account with which the user device 2 is registered.

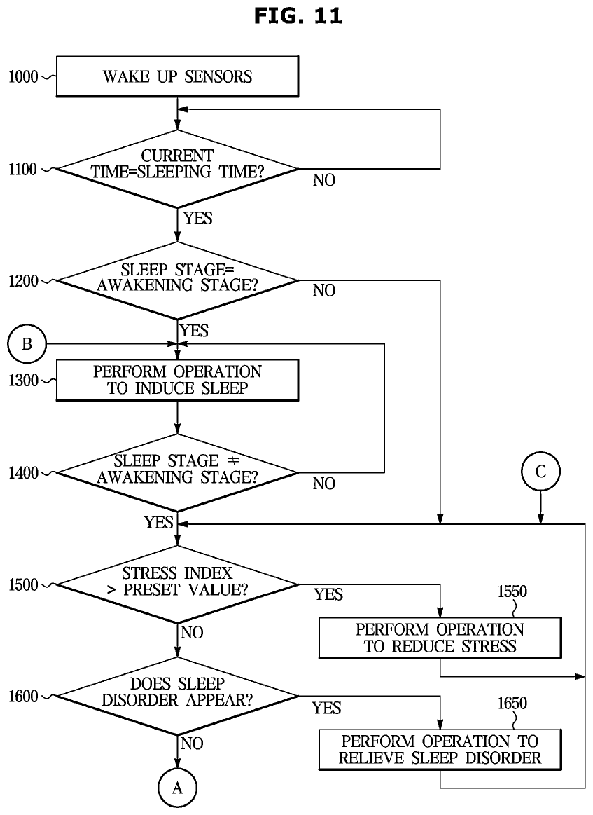

A procedure S6 for generating the control command to control the home appliance 4 based on the sleep state information will be described in detail later with reference to FIGS. 11 to 16. The server device 3 may transmit the control command to control the home appliance 4 to the home appliance 4, in S7.

In one or more embodiments, it is obvious that the procedure S6 for generating the control command to control the home appliance 4 based on the sleep state information and the procedure for transmitting the control command to control the home appliance 4 to the home appliance 4 may be performed by the user device 2 as well.

The home appliance 4 may perform a preset operation corresponding to the control command received from the server device 3, in S8.

The machine learning model stored in the user device 2 may be updated by an external server. For this, in one or more embodiments, data output by the at least one of machine learning models (the first machine learning model 11, the second machine learning model 12, the third machine learning model 13, the fourth machine learning model 14, or the fifth machine learning model 15) installed in the hub device 1 may be sent to the server device 3.

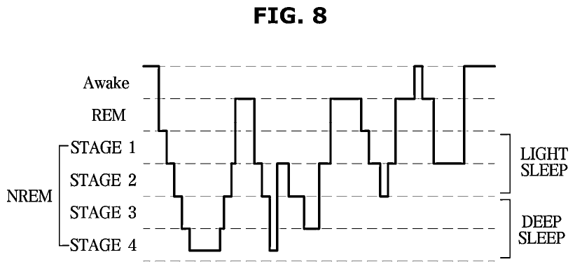

FIG. 8 is a diagram for describing sleep stages.

Sleep stages of humans may be classified into an awakening stage, a rapid eye movement (REM) sleep stage and a non-REM (NREM) sleep stage.

The awakening stage corresponds to a stage in which a person is awake.

The REM sleep stage is a rapid eye movement sleep stage, which corresponds to a shallow sleep close to being awake, and is a stage of sleep distinguished by a rapid eye movement.

REM sleep of adults generally occurs at about 20 to 25% of the total amount of sleep. It repeats for about 90 to 120 minutes of nighttime sleep.

Brain's neural activity during the REM sleep is quite similar to when it is awake. However, the human body is in a relaxed state and thus becomes paralyzed. For this reason, the REM sleep stage is called paradoxical sleep. This means that brain waves are not suppressed during the REM sleep.

The NREM sleep stage is a non-rapid eye movement sleep stage, and unlike the REM sleep, there is little eye movement in the NREM sleep stage.

One seldom dreams during the NREM sleep stage, and muscle movement is not suppressed as in REM sleep. People who do not properly go through the sleep stages may have sleep disorders (e.g., sleepwalking) because they are confined in NREM sleep and their muscles are not suppressed.

The NREM sleep stage may be divided into stage 1 (N1 stage), stage 2 (N2 stage), stage 3 (N3 stage) and stage 4 (N4 stage). In the NREM sleep stage, stage 1 (N1 stage) and stage 2 (N2 stage) may be classified as a light sleep stage, and stage 3 (N3 stage) and stage 4 (N4 stage) may be classified as a deep sleep stage.

In another example, the NREM sleep stage may be divided into stage 1 (N1 stage), stage 2 (N2 stage) and stage 3 (N3 stage).

In the NREM sleep stage, brainwave activity gradually slows down and the physiological function declines. During the NREM sleep stage, brain tissue cells and epithelial cells are regenerated, body energy is restored, and hormones for skeletal growth, protein synthesis and tissue regeneration are secreted.

The N1 stage is the most borderline phase in the sleep state, indicating a procedure in which human body slowly falls asleep. The N1 stage appears before falling into a deep sleep, and refers to a state of not falling into a deep sleep.

The N2 stage is a phase that goes into a deeper sleep from the borderline sleep phase. The brain wave pattern changes from slightly slow beta brain waves to slower theta brain waves, and eye movements stop and intermittent fast eye movements do not appear.

The N3 stage is an early stage of deep sleep, where the brainwave patterns change into delta brainwaves and muscle tension is relaxed so that there is little physical motion. In the N3 stage, snoring symptoms may appear.

The N4 stage corresponds to a deep sleep stage where it is very difficult to be awake. In the N4 stage, secretion of hormones for skeletal growth, protein synthesis and tissue regeneration may increase, and sleepwalking, bed-wetting, etc., may appear.

It is common for the first sleep cycle to begin when the person starts to fall asleep. In the first sleep cycle, the person enters into the NREM sleep stage from the awakening stage, goes through the N1, N2, N3 and N4 stages, and goes back to the N3, N2, N1, and REM sleep stages.

Subsequently, in the second sleep cycle, the person goes through the N1, N2, N3 and N4 stages and goes back to the N3 and N2 stages.

Subsequently, in the third sleep cycle, the person goes through the N3 stage and goes back to the N2, N1 and REM sleep stages.

Subsequently, in the fourth sleep cycle, the person enters back into the N1 and REM sleep stages after going through the N1 and N2 stages.

The person then naturally wakes up while going through the N1, N2 and REM sleep stages.

When the person wakes up during the N4 stage, he/she may become groggy as if he/she did not sleep even after getting out of bed. Hence, the person may need to wake up during the N1 stage or REM sleep stage.

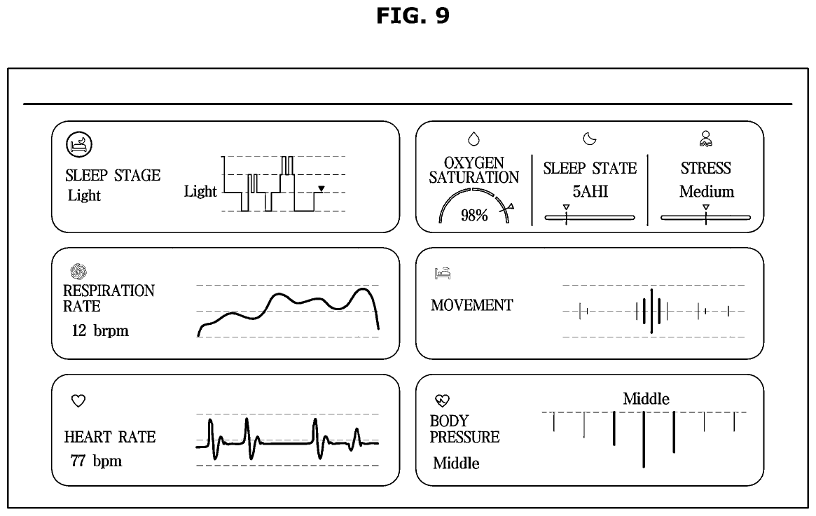

FIG. 9 illustrates an example of sleep state information derived by a sleep management system, according to an embodiment.

The sleep state information may be derived by the sleep management system in real time.

Referring to FIG. 9, the sleep state information may be derived by the sleep management system may include information about a sleep stage, information about oxygen saturation, information about sleep disorder, information about a stress index, information about a respiration rate, information about a movement, information about a heart rate and/or information about body pressure.

In one embodiment, the user device 2 (or the display device 41) may output the sleep state information through the output interface (e.g., a display). For example, based on obtaining of the sleep state information by the user device 2, the information about a sleep stage may include information about a current sleep stage of the user and/or information about the user's sleep stages over time.

The information about the current sleep stage of the user may include information indicating which one of awakening stage, the REM sleep stage or the stage 1, stage 2 or stage 3 of NREM sleep the current sleep stage of the user corresponds to.

The information about the user's sleep stages over time may include information about changes in sleep stage from a time preset by the user with the user device 2, a time when the user lies or sits on the furniture 10 to fall asleep, a time when the user falls asleep and/or a time when the user runs the sleep management application to the current time.

When the information about the user's sleep stages over time is output by the user device 2 (or the display device 41), the information about the user's sleep stages over time may be represented in the form of a graph having the X-axis indicating time and the Y-axis indicating the user's sleep stages.

The information about oxygen saturation may include information about current oxygen saturation of the user and/or information about the user's oxygen saturation over time.

When the information about oxygen saturation is output by the user device 2 (or the display device 41), numerical values of the oxygen saturation may be output in percentage. Furthermore, when the information about oxygen saturation is output by the user device 2 (or the display device 41), whether the oxygen saturation of the user is normal according to medical standards may be displayed.

The information about sleep disorder may include information about a current sleep disorder of the user and/or information about the user's sleep disorders that appear over time.

The sleep disorders may include information about sleep disorder related to respiration such as apnea and hypopnea. The information about sleep disorder related to respiration such as apnea and hypopnea may include an apnea hypopnea index (AHI).

When the information about sleep disorders related to respiration is output by the user device 2 (or the display device 41), the AHI may be output with an indication whether the user's AHI is normal according to the medical standard.

The information about a stress index may include information about a stress level of the user. When the information about a stress index is output by the user device 2 (or the display device 41), the user's stress index may be output in a numerical value or in the form of a comparative word (e.g., high, medium, and low) with an indication whether the user's stress index is normal according to the medical standard.

The information about a respiration rate may include information about a current respiration rate of the user and/or information about the user's respiration rates over time.

When the information about the current respiration rate is output by the user device 2 (or the display device 41), the user's respiration rate may be output in a numerical value with an indication whether the user's respiration rate is normal according to the medical standard.

When the information about the user's respiration rates over time is output by the user device 2 (or the display device 41), the information about the user's respiration rates over time may be represented in the form of a graph having the X-axis indicating time and the Y-axis indicating the user's respiration rates.

The information about movement may include information about a degree of the user's movement and/or information about the user's movement degrees over time.

When the information about the current movement degree is output by the user device 2 (or the display device 41), the user's movement degree may be output in a numerical value or in the form of a comparative word (e.g., high, medium, and low).

When the information about movement degrees over time is output by the user device 2 (or the display device 41), the information about the user's movement degrees over time may be represented in the form of a graph having the X-axis indicating time and the Y-axis indicating the user's movement degrees.

The information about heart rate may include information about a current heart rate of the user and/or information about the user's heart rates over time.

When the information about the current heart rate is output by the user device 2 (or the display device 41), the user's heart rate may be output in a numerical value with an indication whether the user's heart rate is normal according to the medical standard.

When the information about the user's heart rates over time is output by the user device 2 (or the display device 41), the information about the user's heart rates over time may be represented in the form of a graph having the X-axis indicating time and the Y-axis indicating the user's heart rates.

The information about body pressure may include information about the user's body posture. The information about body posture may include information regarding whether the user's posture corresponds to lying on one's back, on the left side, on the right side, or on one's face, sitting up, etc.

When the information about body pressure is output by the user device 2 (or the display device 41), a term representing the user's posture may be used, and the form of a pressure distribution table based on the furniture 10 on which the user lies or sits may be output.

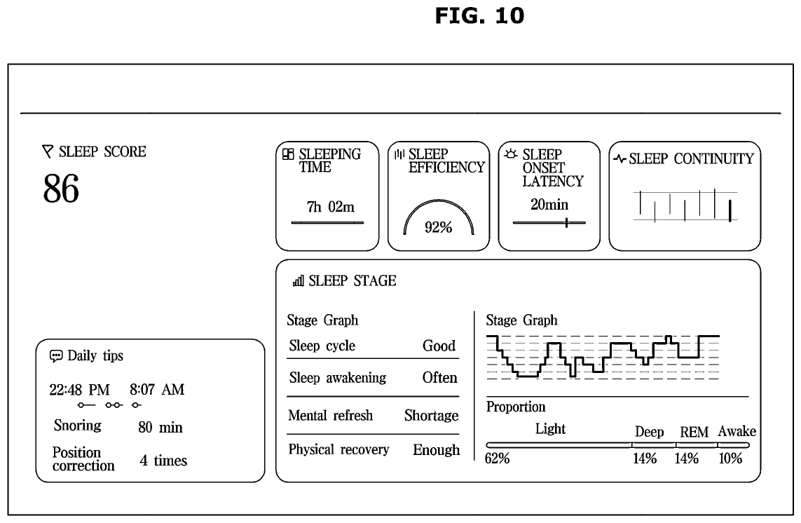

FIG. 10 illustrates an example of sleep summary information derived by a sleep management system, according to an embodiment.

The sleep summary information may include a collection of sleep state information obtained by the sleep management system in real time.

For example, the sleep summary information may include sleep score information, sleep time information, sleep efficiency information, sleep onset latency information, sleep continuity information, and/or sleep stage information corresponding to sleep quality.

The sleep score corresponding to sleep quality may be determined based on the sleep state information obtained in real time during the user's sleep. For example, the user device 2 and/or the server device 3 may calculate the sleep score based on the sleep state information obtained in real time during the user's sleep. Information about the sleep score may be calculated based on the sleep stage, oxygen saturation, sleep disorder, stress index, respiration rate, movement, heart rate and/or body pressure during the user's sleep.

The sleep time information may include information about a time from when the user goes through a change from the awakening state to a sleep state to when the user finally wakes up.

In another example, the sleep time information may include information about a time from when the person lies on the furniture 10 to when he/she finally wakes up.

The information about sleep efficiency may be determined based on a time proportion of a preset stage (e.g., the NREM sleep stage) in the sleep stage. The user device 2 and/or the server device 3 may calculate sleep efficiency based on the sleep state information obtained in real time during the user's sleep. In an embodiment, the user device 2 and/or the server device 3 may determine sleep efficiency based on a time proportion of a preset stage (e.g., the NREM sleep stage) in the user's sleep stage.

The information about the sleep onset latency (SOL) may include information about a time required for the sleep stage to be changed from the awakening stage to the NREM sleep stage.

The information about the sleep continuity may be determined based on a time for which the sleep phase does not change into the awakening phase (and/or the REM sleep stage) but the NREM sleep stage is maintained. The user device 2 and/or the server device 3 may determine sleep continuity based on the sleep state information obtained in real time during the user's sleep. In an embodiment, the user device 2 and/or the server device 3 may determine sleep continuity based on a time for which a preset stage (e.g., the NREM sleep stage) among the user's sleep stage is maintained.

The information about the sleep stage may include information about changes in sleep stage over time. The information about the sleep stage may include information about a time proportion of each sleep stage during the user's sleep, information about whether the user's sleep cycle corresponds to a normal range, and information about an extent to which the body is repaired, an extent to which the brain is repaired and/or an extent of periodic awakening determined based on a time proportion of each sleep phase during the user's sleep.

The sleep summary information is not limited to the above example, and may include various information associated with the user's sleep. For example, the sleep summary information may include information about the user's snoring time and information about the number of changes in the user's posture.

The sleep state information and the sleep summary information as described with reference to FIGS. 9 and 10 may be provided for the user by the user device 2 or by the home appliance 4 (e.g., the display device 41, the speaker 46, etc.).

For example, the user may use the user device 2 to run a sleep management application, and check the sleep state information and the sleep summary information through an interface provided by the sleep management application.

In another example, the server device 3 may control the home appliance 4 (e.g., the display device 41, the speaker 46, etc.) to provide the sleep summary information when the user wakes up from the sleep.

In one or more embodiments, the user may share the sleep state information and/or sleep summary information with other users through the user device 2. For example, the user may run the sleep management application, and transmit the sleep state information and/or the sleep summary information to the other users' devices through the interface provided by the sleep management application.

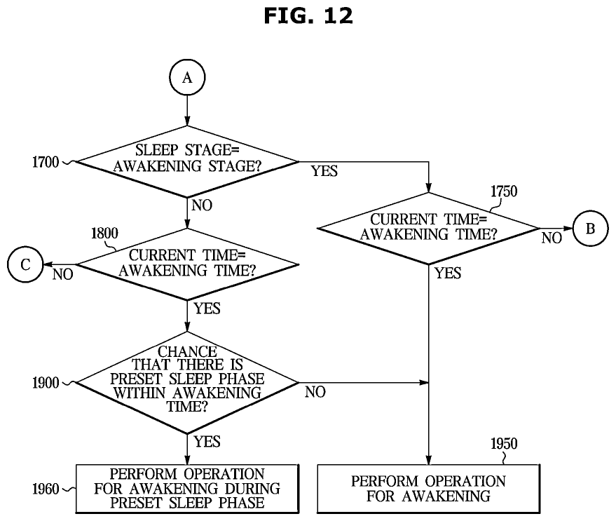

FIGS. 11 and 12 illustrate an example of a flowchart of a sleep management method, according to an embodiment.

As described above with reference to FIG. 6, in an embodiment, the server device 3 may control the home appliance 4 based on the sleep state information.

In an embodiment, the server device 3 may control the home appliance 4 based on a control command received from the user device 2.

As described above, the user device 2 may generate the control command by itself based on the sleep state information, or the server device 3 may generate the control command based on the sleep state information, or both the user device 2 and the server device 3 may generate the control command based on the sleep state information.

Accordingly, it is obvious that operations, as will be described below, performed by the server device 3 may be performed by the user device 2 as well. The at least one memory 220 in the user device 2 may store instructions that enable the at least one processor 210 to perform the aforementioned and the following operations.

Referring to FIGS. 11 and 12, the hub device 1 may wake up the plurality of sensors 5 based on a preset condition being satisfied.

For example, the hub device 1 may wake up the plurality of sensors 5 based on determining that there is a user on the furniture 10.

In another example, the user device 2 may transmit a request to start sleep management to the hub device 1 based on receiving of a user input corresponding to start of sleep, and the hub device 1 may wake up the plurality of sensors 5 based on receiving of the request to start sleep management through the user device 2.

In another example, the user device 2 may receive information about an expected sleep time from the user and forward the information to the hub device 1. The hub device 1 may wake up the plurality of sensors 5 based on reaching the expected sleep time.

The server device 3 may determine whether the current time corresponds to a sleeping time, in 1100.

For example, the user may preset the sleeping time through the user device 2. The sleeping time may be set to a time range or a time. For example, the sleeping time may be set by the user to “10 p.m. to 11 p.m., or set to “10 p.m.”.

The server device 3 may determine that the current time corresponds to the sleeping time when the current time corresponds to the sleeping time set by the user.

In another example, when the user inputs a command corresponding to start of sleep through the user device 2, the server device 3 may determine that the current time corresponds to the sleeping time.

The command corresponding to start of sleep may be received through the user interface 240 of the user device 2. For example, the command corresponding to start of sleep may be received by the user device 2 through a touch input and/or sound input.

In one or more embodiments, when the user inputs an automatic sleep management request through the user device 2, the server device 3 may determine that the current time corresponds to the sleeping time based on a preset condition being satisfied. In an embodiment, the server device 3 may determine whether the current time corresponds to the sleeping time based on the resultant information. For example, the server device 3 may determine that the current time corresponds to the sleeping time when the user's preset posture (e.g., posture of lying on the back or on the face) is maintained for a preset period of time.

When the current time corresponds to the sleeping time in 1100, the server device 3 may control the home appliance 4 based on the sleep state information received from the user device 2.

In an embodiment, when the current time corresponds to the sleeping time in 1100 and the user's sleep stage corresponds to a preset stage (e.g., awakening stage) in 1200, the server device 3 may control the home appliance 4 to perform a preset operation to induce sleep in 1300.

For example, the user device 2 may transmit a request for start of sleep to the server device 3 based on receiving of a command corresponding to start of sleep from the user, and the server device 3 may receive the request for the start of sleep, and control the home appliance 4 to perform a preset operation to induce sleep when the user's sleep stage corresponds to a preset stage (e.g., awakening stage).

The preset operation to induce sleep may include a preset operation to be performed by each of the at least one home appliance 4.

The preset operation to induce sleep may be set in advance, and may be changeable by the user through the user device 2.

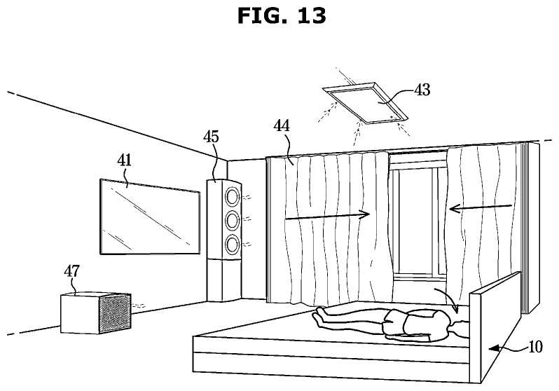

FIG. 13 illustrates an example of a preset operation to induce sleep of a user.

Referring to FIG. 13, the server device 3 may control the display device 41 to perform the preset operation to induce sleep.

For example, the server device 3 may control the display device 41 so that the brightness of an image output from the display device 41 gradually becomes darker. In another example, the display device 41 may be controlled to play preset music (e.g., music to induce sleep).

In one or more embodiments, the server device 3 may control the speaker 46 to play certain music (e.g., music to induce sleep).

The server device 3 may control the lighting device 43 to perform a preset operation to induce sleep. For example, the server device 3 may control the lighting device 43 so that the brightness of light output from the lighting device 43 gradually becomes darker. In another example, the server device 3 may control the lighting device 43 so that color of the light output from the lighting device 43 is changed to a preset color (e.g., a color having a color temperature of light of 2000K or less).

The server device 3 may control the automatic curtain open/close device 44 to perform a preset operation to induce sleep. For example, the server device 3 may control the automatic curtain open/close device 44 to close the curtain.

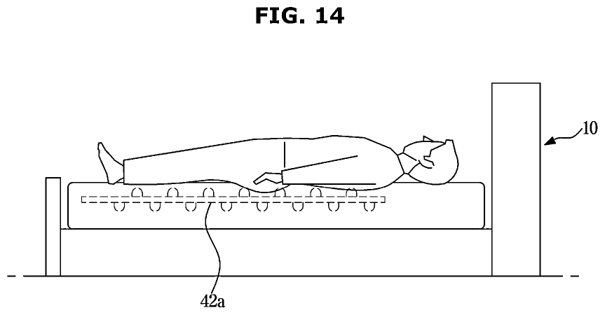

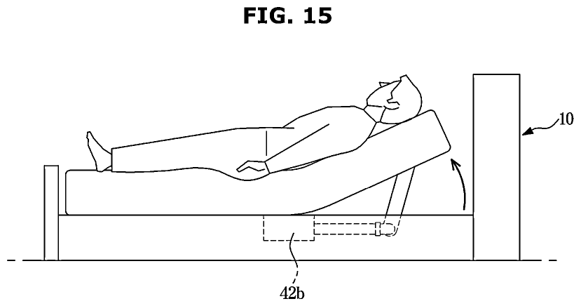

The furniture control device 42 may include a vibration element 42a that is able to transmit vibration to the user who lies or sits on the furniture 10 and/or an actuator 42b that is able to change the posture of the user by changing the structure of the furniture 10 (see FIGS. 14 and 15).

The server device 3 may control the furniture control device 42 to perform a preset operation to induce sleep.

For example, the server device 3 may control the vibration element 42a to output vibration at preset intervals to ease the tension of the user.

In another example, the server device 3 may control the actuator 42b to change the structure of the furniture so that the user's posture is changed to a preset posture (lying on his/her back) that makes it easy to fall sleep.

The server device 3 may control the air conditioner 45 and/or the air purifier 47 to perform a preset operation to induce sleep. For example, the server device 3 may control the air conditioner and/or the air purifier 47 to operate in a sleep mode.

While operating in the sleep mode, the air conditioner 45 and/or the air purifier 47 may minimize noise, for example, by controlling the fan speed to slow down.

The preset operation to induce sleep that may be performed by the home appliance 4 is not limited to the above example, and may be obviously changed according to the user's setting and the type of the home appliance 4.

Turning back to FIG. 11, the server device 3 may determine whether the user's sleep stage corresponds to the awakening stage, in 1400.

When the user's sleep stage does not correspond to the awakening stage in 1400, the server device 3 may control the home appliance 4 to perform a preset operation to reduce the user's stress index or relieve the sleep disorder.

In an embodiment, when the user's sleep stage corresponds to the REM sleep stage or NREM sleep stage, the server device 3 may control the home appliance 4 to perform a preset operation to reduce the user's stress index or relieve the sleep disorder.

In some embodiments, only when the user's sleep stage corresponds to the NREM sleep stage, the server device 3 may control the home appliance 4 to perform a preset operation to reduce the user's stress index or relieve the sleep disorder.

According to an embodiment of the disclosure, by controlling the home appliance 4 to perform a preset operation to reduce the user's stress index or relieve the sleep disorder only when the user's sleep stage corresponds to the NREM sleep stage, the user may be prevented from waking up during the REM sleep stage due to an operation of the home appliance 4.

The preset operation to reduce stress that may be performed by the home appliance 4 is not limited to the above example, and may be obviously changed according to the user's setting and the type of the home appliance 4.

The server device 3 may control the home appliance 4 in 1550 to perform the preset operation to reduce the user's stress in response to the user's stress index exceeding a preset value in 1500.

FIG. 14 illustrates an example of a preset operation to relieve stress of a user.

Referring to FIG. 14, the server device 3 may control the lighting device 43 to perform the preset operation to reduce the user's stress.

For example, the server device 3 may control the lighting device 43 to output light of a preset color (e.g., a color having a color temperature of light of 1000K or less).

The server device 3 may control the air conditioner 45 to perform the preset operation to reduce the user's stress.

For example, the server device 3 may control the air conditioner 45 to slightly increase the fan speed so that the wind output from the air conditioner 45 may reach the user.

The server device 3 may control the furniture control device 42 to perform a preset operation to reduce the user's stress. The furniture control device 42 may include the vibration element 42a that is able to transmit vibration to the user who lies or sits on the furniture 10.

For example, the server device 3 may control the vibration element 42a to output vibration corresponding to the user's heart rate.

In an embodiment of the disclosure, by reducing the user's stress index during the sleep, quality sleep of the user may be induced.

Turning back to FIG. 11, in response to the appearance of a preset sleep disorder to the user in 1600, the server device 3 may control the home appliance 4 to perform a preset operation to relieve the sleep disorder that appears, in 1650.

The preset operation to relieve the sleep disorder that may be performed by the home appliance 4 is not limited to the above example, and may be obviously changed according to the user's setting and the type of the home appliance 4.

FIG. 15 illustrates an example of a preset operation to relieve sleep disorders of the user.

Referring to FIG. 15, the server device 3 may control the air conditioner 45 to perform a preset operation to relieve the sleep disorder.

For example, the server device 3 may turn off the air conditioner 45.

The server device 3 may control the furniture control device 42 to perform a preset operation to relieve the sleep disorder. The furniture control device 42 may include the actuator 42b that is able to change the user's posture by changing the structure of the furniture 10.

For example, the server device 3 may control the actuator 42b to change the structure of the furniture so that the user's posture is changed to a preset posture (in which the user's head is located higher than the torso and leg portions) that eases the user's breathing.

In an embodiment of the disclosure, by relieving the sleep disorder that appears during the sleep, quality sleep of the user may be induced.

Turning back to FIG. 12, the server device 3 may control the home appliance 4 to perform a preset operation for awakening in 1950, based on the sleep stage not corresponding to the awakening stage in 1700 and the current time corresponding to awakening time in 1800. For example, the user may preset the awakening time through the user device 2.

The awakening time may be set to a time range or a time. For example, the awakening time may be set by the user to “7 a.m. to 8 a.m., or set to “7 a.m.”.

The server device 3 may determine that the current time corresponds to the awakening time when the current time corresponds to the awakening time set by the user.

In another example, when the user inputs a command corresponding to start of awakening through the user device 2, the server device 3 may determine that the current time corresponds to the awakening time.

The command corresponding to the start of awakening may be received through the user interface 240 of the user device 2. For example, the command corresponding to the start of awakening may be received by the user device 2 through a touch input and/or sound input.

In one or more embodiments, when the user inputs an automatic sleep management request through the user device 2, the server device 3 may determine that the current time corresponds to the awakening time based on a preset condition being satisfied. In an embodiment, the server device 3 may determine whether the current time corresponds to the awakening time based on the resultant information. For example, the server device 3 may determine that the current time corresponds to the awakening time when the user's sleep stage is maintained for a preset period of time (e.g., 10 minutes) as the awakening stage.

As described above, the person may need to wake up during the N1 stage or REM sleep stage.

The server device 3 may determine a chance that a preset sleep stage (e.g., the REM sleep stage and/or the NREM sleep stage) enters the N1 stage within the awakening time based on the sleep state information, in 1900.

In a case that the awakening time is set to a time range, the determining of the chance of entering a preset sleep stage (e.g., the REM sleep stage and/or the N1 stage of the NREM sleep stage) within the awakening time may include determining whether the user's sleep stage is going to enter the preset sleep stage (e.g., the REM sleep stage and/or the N1 stage of the NREM sleep stage) within the set time range.

In a case that the awakening time is set to a time, the determining of the chance of entering a preset sleep stage (e.g., the REM sleep stage and/or the N1 stage of the NREM sleep stage) may include determining whether the user's sleep stage is going to enter the preset sleep stage (e.g., the REM sleep stage and/or the N1 stage of the NREM sleep stage) within a preset time (e.g., 15 minutes) around the time corresponding to the awakening time.

When there is a chance that the user's sleep stage is going to enter the preset stage (e.g., the REM sleep stage and/or the N1 stage of the NREM sleep stage) within the awakening time in 1900, the server device 3 may control the home appliance 4 to perform a preset operation to wake up the user based on the user's sleep stage entering the preset stage (e.g., the REM sleep stage and/or the N1 stage of the NREM sleep stage) in 1960.

In other words, the server device 3 may control the home appliance 4 to perform the preset operation to wake up the user when the current time corresponds to the awakening time and the user's sleep stage corresponds to the preset stage (e.g., the REM sleep stage and/or the N1 stage of the NREM sleep stage).

In the disclosure, by waking up the user during the REM sleep stage and/or the N1 stage of the NREM stage, the user may be led to feel fresh when he/she is awakened.

On the other hand, when there is no chance that the user's sleep stage is going to enter the preset stage (e.g., the REM sleep stage and/or the N1 stage of the NREM sleep stage) within the awakening time in 1900, and when the current time corresponds to the awakening time, the server device 3 may control the home appliance 4 to perform a preset operation to wake up the user in 1950.

In an embodiment, the server device 3 may control the home appliance 4 to perform a preset operation for waking up the user in 1950, when the user's sleep stage corresponds to the awakening stage and the current time corresponds to the awakening time in 1750.

In an embodiment, the server device 3 may perform the operation for awakening or perform an operation to induce sleep depending on the user input, when the user's sleep stage corresponds to the awakening stage and the current time does not correspond to the awakening time in 1750.

For example, when the current time in the user's sleep stage does not correspond to the awakening time, the server device 3 may perform the operation for awakening in 1950, based on the user device 2 receiving a user input to request the start of awakening.

In another example, when the current time in the user's sleep stage does not correspond to the awakening time, the server device 3 may perform the operation to induce sleep, based on the user device 2 failing to receive the user input to request the start of awakening within a preset time, in 1300.

In another example, when the current time in the user's sleep stage does not correspond to the awakening time, the server device 3 may perform the operation to induce sleep based on the user device 2 receiving a user input to request to start to sleep again in 1300.

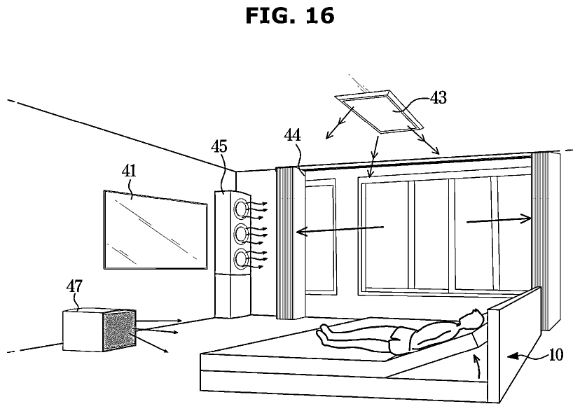

FIG. 16 illustrates an example of a preset operation to induce wakeup of the user.

Referring to FIG. 16, the server device 3 may control the display device 41 to perform the preset operation to induce sleep.

For example, the server device 3 may control the display device 41 so that the brightness of an image output from the display device 41 gradually becomes brighter. In another example, the display device 41 may be controlled to play preset music (e.g., music to induce wakeup).

In one or more embodiments, the server device 3 may control the speaker 46 to play preset music (e.g., music to induce wakeup).

The server device 3 may control the lighting device 43 to perform a preset operation to induce wakeup. For example, the server device 3 may control the lighting device 43 so that the brightness of light output from the lighting device 43 gradually becomes brighter. In another example, the server device 3 may control the lighting device 43 to output light similar to natural light.

The server device 3 may control the automatic curtain open/close device 44 to perform a preset operation to induce wakeup. For example, the server device 3 may control the automatic curtain open/close device 44 to open the curtain.

The server device 3 may control the furniture control device 42 to perform a preset operation to induce wakeup. The furniture control device 42 may include the vibration element 42a that is able to transmit vibration to the user who lies or sits on the furniture 10 and/or the actuator 42b that is able to change the posture of the user by changing the structure of the furniture 10.

For example, the server device 3 may control the vibration element 42a to output vibration at preset intervals corresponding to the user's heart rate.

In another example, the server device 3 may control the actuator 42b to change the structure of the furniture so that the user's posture is changed to a preset posture (in which the upper body is located higher than the lower body) that makes it easy for the user to wake up.

The server device 3 may control the air conditioner 45 and/or the air purifier 47 to perform a preset operation to induce wakeup. For example, the server device 3 may control the air conditioner and/or the air purifier 47 to operate in an awakening mode.

While operating in the awakening mode, the air conditioner 45 and/or the air purifier 47 may control the fan at high speed, giving pleasant feeling to the user.

The preset operation to induce wakeup that may be performed by the home appliance 4 is not limited to the above example, and may be obviously changed according to the user's setting and the type of the home appliance 4.

In one or more embodiments, the operations as described above with reference to FIGS. 11 to 16 may be performed by the user device 2 as well. For example, the user device 2 may control the home appliance 4 based on the sleep state information.

According to an embodiment of the disclosure, the user's quality sleep may be induced.

According to an embodiment of the disclosure, a sleep management system may include the plurality of sensors 5 (including the first sensor 51, the second sensor 52, the third sensor 53, the fourth sensor 54, or the fifth sensor 55) for collecting data of a user; the hub device 1 configured to receive first data collected by the first sensor 51 and second data collected by the second sensor 52, obtain first processed data by processing the first data, and obtain second processed data by processing the second data; the user device 2 configured to receive the first processed data and the second processed data from the hub device 1, and obtain sleep state information associated with the user's sleep state by inputting the first processed data and the second processed data to a machine learning model; and the server device 3 configured to receive the sleep state information from the user device 2 by wireless communication.

The hub device 1 may obtain the first processed data by inputting the first data to the first machine learning model 11 (or the second machine learning model 12, the third machine learning model 13, the fourth machine learning model 14, or the fifth machine learning model 15) and obtain the second processed data by inputting the second data to the second machine learning model 12 (or the first machine learning model 11, the third machine learning model 13, the fourth machine learning model 14, or the fifth machine learning model 15).

The hub device 1 may receive data of the user from the plurality of sensors 5 by wired communication.

The hub device 1 may receive the first data from a sensor (e.g., the first sensor 51, the second sensor 52, or the third sensor 53) by wired communication and receive the second data from a sensor (e.g., the fourth sensor 54 or the fifth sensor 55) by wireless communication.

The plurality of sensors 5 may include at least two of a pressure sensor, a UWB sensor, an oxygen saturation sensor, an ECG sensor, or an acceleration sensor.

The sleep state information associated with the user's sleep state may include at least one of information about the user's sleep stage, information about the user's stress index, or information about the user's sleep disorder.

The user device 2 may store a sleep management application including the machine learning model 21.

The sleep management application may be downloadable from an external server.

The server device 3 may control the at least one home appliance connected and registered with the user device 2 based on the sleep state information.

The server device 3 may control the at least one home appliance 4 to perform a preset operation to wake up the user, in response to (or based on) a current time (corresponding to an awakening time) and the user's sleep stage (corresponding to a preset stage).

The server device 3 may control the at least one home appliance 4 to perform a preset operation to induce sleep of the user, in response to (or based on) a current time (corresponding to a sleeping time) and the user's sleep stage (corresponding to a preset stage).

The server device 3 may control the at least one home appliance 4 to perform a preset operation to relieve the user's stress in response to the user's stress index exceeding a preset value.

The server device 3 may control the at least one home appliance 4 to perform a preset operation to relieve preset sleep disorder in response to the preset sleep disorder appearing to the user.

The hub device 1 may include a printed circuit board wiredly connected to the plurality of sensors 5.

According to an embodiment of the disclosure, a sleep management method may include receiving, by the hub device 1, first data collected by the first sensor 51 (or the second sensor 52, the third sensor 53, the fourth sensor 54, or the fifth sensor 55) and second data collected by the second sensor 52 (or the first sensor 51, the third sensor 53, the fourth sensor 54, or the fifth sensor 55); obtaining, by the hub device 1, first processed data by processing the first data and second processed data by processing the second data; transmitting, by the hub device 1, the first processed data and the second processed data to the user device 2; obtaining, by the user device 2, sleep state information associated with a sleep state of the user by inputting the first processed data and the second processed data received from the hub device 1 to a machine learning model; and transmitting, by the user device 2, the sleep state information to the server device 3 by wireless communication.

The obtaining of the first processed data and the second processed data by the hub device 1 may include obtaining the first processed data by inputting the first data to the first machine learning model 11 (or the second machine learning model 12, the third machine learning model 13, the fourth machine learning model 14, or the fifth machine learning model 15) and obtaining the second processed data by inputting the second data to a second machine learning model 12 (or the first machine learning model 11, the third machine learning model 13, the fourth machine learning model 14, or the fifth machine learning model 15).

The receiving of the first data and the second data by the hub device 1 may include receiving the first data from the first sensor 51 (or the second sensor 52 or the third sensor 53) by wired communication and receiving the second data from the second sensor 52 (or the first sensor 51 or the third sensor 53) by wired communication.

The receiving of the first data and the second data by the hub device 1 may include receiving the first data from the first sensor 51, 52 or 53 by wired communication and receiving the second data from the second sensor 54 or 55 by wireless communication.

The sleep management method may further include controlling, by the server device 3, the at least one home appliance connected and registered with the user device 2 based on the sleep state information.

The controlling of the at least one home appliance 4 by the server device 3 may include controlling the at least one home appliance 4 to perform a first preset operation to wake up the user in response to (or based on) a current time corresponding to an awakening time and the user's sleep stage corresponding to a first preset stage; controlling the at least one home appliance 4 to perform a second preset operation to induce sleep of the user in response to (or based on) a current time corresponding to a sleeping time, and the user's sleep stage corresponding to a second preset stage; controlling the at least one home appliance 4 to perform a third preset operation to relieve the user's stress in response to the user's stress index exceeding a preset value; or controlling the at least one home appliance 4 to perform a fourth preset operation to relieve preset sleep disorder in response to the preset sleep disorder appearing to the user.

According to an embodiment of the disclosure, the user device 2 may include the communicator 230 communicating with the hub device 1 for processing data of a user collected from the plurality of sensors 5; the at least one memory 220; and the at least one processor 210 electrically connected to the communicator 230 and the at least one memory 220, wherein the at least one memory 220 is configured to store instructions, which enable, when executed by the at least one processor 210, the at least one processor 210 to receive processed data processed by the hub device 1 through the communicator 230 and obtain sleep state information associated with a sleep state of the user by inputting the processed data to a machine learning model.

The instructions may enable, when executed by the at least one processor 210, the at least one processor 210 to control the at least one home appliance 4 based on the sleep state information.

The instructions may enable, when executed by the at least one processor 210, the at least one processor 210 to control the at least one home appliance 4 to perform a preset operation to wake up the user in response to (or based on) a current time corresponding to an awakening time, and the user's sleep stage corresponding to a preset stage.

The instructions may enable, when executed by the at least one processor 210, the at least one processor 210 to control the at least one home appliance 4 to perform a preset operation to wake up the user in response to (or based on) a current time corresponding to an awakening time, and the user's sleep stage corresponding to a preset stage.

The instructions may enable, when executed by the at least one processor 210, the at least one processor 210 to control the at least one home appliance 4 to perform a preset operation to induce sleep of the user in response to (or based on) a current time corresponding to a sleeping time, and the user's sleep stage corresponding to an awakening stage.

The instructions may enable, when executed by the at least one processor 210, the at least one processor 210 to control the at least one home appliance 4 to perform a preset operation to relieve the user's stress in response to the user's stress index exceeding a preset value.

The instructions may enable, when executed by the at least one processor 210, the at least one processor 210 to control the at least one home appliance 4 to perform a preset operation to relieve preset sleep disorder in response to the preset sleep disorder appearing to the user.

The instructions may enable, when executed by the at least one processor 210, the at least one processor 210 to transmit the sleep state information to the server 3 through the communicator 230.

The instructions may enable, when executed by the at least one processor 210, the at least one processor 210 to transmit a control command to control the at least one home appliance 4 to the server 3 through the communicator 230.

The instructions may enable, when executed by the at least one processor 210, the at least one processor 210 to output the sleep state information through the user interface 240.

According to an embodiment of the disclosure, a non-transitory storage medium is configured to store computer-readable instructions, wherein the instructions may enable, when executed by the processor 210, the processor 210 to receive processed data processed by the hub device 1 from the hub device 1 for processing data of a user collected from the plurality of sensors 5 through the communicator 230; and obtain sleep state information associated with a sleep state of the user by inputting the processed data to a machine learning model.

The instructions may enable, when executed by the processor 210, the processor 210 to control the at least one home appliance 4 based on the sleep state information.

The instructions may enable, when executed by the processor 210, the processor 210 to control the at least one home appliance 4 to perform a preset operation to wake up the user in response to (or based on) a current time corresponding to an awakening time and the user's sleep stage corresponding to a preset stage.

The instructions may enable, when executed by the processor 210, the processor 210 to control the at least one home appliance 4 to perform a preset operation to wake up the user in response to (or based on) a current time corresponding to an awakening time and the user's sleep stage corresponding to a preset stage.

The instructions may enable, when executed by the processor 210, the processor 210 to control the at least one home appliance 4 to perform a preset operation to induce sleep of the user in response to (or based on) a current time corresponding to a sleeping time and the user's sleep stage corresponding to an awakening stage.

The instructions may enable, when executed by the processor 210, the processor 210 to control the at least one home appliance 4 to perform a preset operation to relieve the user's stress in response to the user's stress index exceeding a preset value.

The instructions may enable, when executed by the processor 210, the processor 210 to control the at least one home appliance 4 to perform a preset operation to relieve preset sleep disorder in response to the preset sleep disorder appearing to the user.

The instructions may enable, when executed by the processor 210, the processor 210 to transmit the sleep state information to the server 3 through the communicator 230.

The instructions may enable, when executed by the processor 210, the processor 210 to transmit a control command to control the at least one home appliance 4 to the server 3 through the communicator 230.

Meanwhile, the embodiments of the disclosure may be implemented in the form of a recording medium for storing instructions to be carried out by a computer. The instructions may be stored in the form of program codes, and when executed by a processor, may generate program modules to perform operations in the embodiments of the disclosure. The recording media may correspond to computer-readable recording media.

The computer-readable recording medium includes any type of recording medium having data stored thereon that may be thereafter read by a computer. For example, it may be a read only memory (ROM), a random access memory (RAM), a magnetic tape, a magnetic disk, a flash memory, an optical data storage device, etc.

The computer-readable storage medium may be provided in the form of a non-transitory storage medium. The term ‘non-transitory storage medium’ may mean a tangible device without including a signal, e.g., electromagnetic waves, and may not distinguish between storing data in the storage medium semi-permanently and temporarily. For example, the non-transitory storage medium may include a buffer that temporarily stores data.

In an embodiment of the disclosure, the aforementioned method according to the one or more embodiments of the disclosure may be provided in a computer program product. The computer program product may be a commercial product that may be traded between a seller and a buyer. The computer program product may be distributed in the form of a recording medium (e.g., a compact disc read only memory (CD-ROM)), through an application store (e.g., Play Store™), directly between two user devices (e.g., smart phones), or online (e.g., downloaded or uploaded). In the case of online distribution, at least part of the computer program product (e.g., a downloadable app) may be at least temporarily stored or arbitrarily created in a recording medium that may be readable to a device such as a server of the manufacturer, a server of the application store, or a relay server.

Several embodiments of the disclosure have been described above, but a person of ordinary skill in the art will understand and appreciate that various modifications can be made without departing from the scope of the disclosure. Thus, it will be apparent to those of ordinary skill in the art that the true scope of technical protection is only defined by the following claims.

What is claimed is:

1. A sleep management system comprising:
a plurality of sensors configured to collect data of a user;
a hub device configured to process the data collected from the plurality of sensors;
a user device configured to obtain sleep state information by processing the data processed by the hub device, wherein the sleep state information is associated with a sleep state of the user; and
a server device configured to control at least one home appliance, based on the sleep state information obtained by the user device,
wherein the plurality of sensors comprise a first sensor configured to collect first data and a second sensor configured to collect second data,
wherein the hub device is further configured to:
receive the first data and the second data,
obtain first processed data by processing the first data,
obtain second processed data by processing the second data, and
transmit the first processed data and the second processed data to the user device, and
wherein the user device is further configured to:
receive the first processed data and the second processed data from the hub device,
obtain the sleep state information by inputting the first processed data and the second processed data to a machine learning model of the user device, and transmit the sleep state information to the server device.

2. The sleep management system of claim 1, wherein the hub device is further configured to:
obtain the first processed data by inputting the first data to a first machine learning model of the hub device, and
obtain the second processed data by inputting the second data to a second machine learning model of the hub device.

3. The sleep management system of claim 1, wherein the hub device is further configured to receive the data of the user from the plurality of sensors by wired communication.

4. The sleep management system of claim 1, wherein the hub device is further configured to:
receive the first data from the first sensor by wired communication, and
receive the second data from the second sensor by wireless communication.

5. The sleep management system of claim 1, wherein the plurality of sensors comprise at least two of a pressure sensor, a UWB sensor, an oxygen saturation sensor, an electrocardiogram sensor, or an acceleration sensor.

6. The sleep management system of claim 1, wherein the sleep state information comprises at least one of information about a sleep stage of the user, information about a stress index of the user, or information about a sleep disorder of the user.

7. The sleep management system of claim 1, wherein each of the first processed data and the second processed data comprises probability values of a sleep stage of the user, and
wherein the machine learning model of the user device is configured to determine the sleep stage of the user by assigning different weights to the probability values of the sleep stage of the user.

8. The sleep management system of claim 1, wherein each of the first processed data and the second processed data comprises values of a stress index of the user, and
wherein the machine learning model of the user device is configured to determine the stress index of the user by assigning different weights to the values of the stress index of the user.

9. The sleep management system of claim 1, wherein each of the first processed data and the second processed data comprises apnea-hypopnea indexes of the user, and
wherein the machine learning model of the user device is configured to determine apnea-hypopnea index of the user by assigning different weights to the apnea-hypopnea indexes of the user.

10. The sleep management system of claim 1, wherein the sleep state information comprises information about a sleep stage of the user, and
wherein the server device is further configured to control the at least one home appliance to perform a preset operation of waking up the user, in response to a current time corresponding to an awakening time and the sleep stage of the user corresponding to a preset stage.

11. The sleep management system of claim 1, wherein the sleep state information comprises information about a sleep stage of the user, and
wherein the server device is further configured to control the at least one home appliance to perform a preset operation of inducing sleep of the user, in response to a current time corresponding to a sleeping time and the sleep stage of the user corresponding to an awakening stage.

12. The sleep management system of claim 1, wherein the sleep state information comprises information about a stress index of the user, and
wherein the server device is further configured to control the at least one home appliance to perform a preset operation of relieving a stress of the user in response to the stress index of the user exceeding a preset value.

13. The sleep management system of claim 1, wherein the sleep state information comprises information about a sleep disorder of the user, and
wherein the server device is further configured to control the at least one home appliance to perform a preset operation of relieving preset sleep disorder in response to an appearance of the preset sleep disorder to the user.

14. A sleep management method comprising:
receiving, by a hub device, first data collected by a first sensor and second data collected by a second sensor;
obtaining, by the hub device, first processed data by processing the first data and second processed data by processing the second data;
transmitting, by the hub device, the first processed data and the second processed data to a user device;
obtaining, by the user device, sleep state information by inputting the first processed data and the second processed data to a machine learning model of the user device, wherein the sleep state information is associated with a sleep state of a user; and
transmitting, by the user device, the sleep state information to a server device.

15. The sleep management method of claim 14, wherein the obtaining, by the hub device, the first processed data by processing the first data and the second processed data by processing the second data comprises obtaining the first processed data by inputting the first data to a first machine learning model of the hub device, and obtaining the second processed data by inputting the second data to a second machine learning model of the hub device.
